(19) United States
(12) Patent Application Publication
(10) Pub. No.: US 2020/0405867 A1
(43) Pub. Date: Dec. 31, 2020

(54) ORAL DOSAGE FORMS

(71) Applicant: Sunovion Pharmaceuticals Inc., Marlborough, MA (US)

(72) Inventors: Maiko ONITA, Chuo-ku, Osaka-shi (JP); Shahla JAMZAD, Belmont, MA (US)

(73) Assignee: Sunovion Pharmaceuticals Inc., Marlborough, MA (US)

(21) Appl. No.: 16/963,116

(22) PCT Filed: Jan. 18, 2019
(86) PCT No.: PCT/US2019/014170

§ 371 Date: Jul. 17, 2020

Related U.S. Application Data
(60) Provisional application No. 62/619,174, filed on Jan. 19, 2018.

(51) Int. Cl. A61K 47/40; A61K 9/00; A61K 31/136; A61K 47/12; A61K 47/26

(52) U.S. Cl. CPC A61K 47/40; A61K 9/0053; A61K 47/26; A61K 47/12; A61K 31/136

(57) ABSTRACT

The present application generally relates to pharmaceutical compositions comprising dasotraline, and methods of using the pharmaceutical compositions thereof to treat disorders, while limiting a perceived bitter taste or paresthesic sensation.

### CROSS-REFERENCE TO RELATED APPLICATIONS

This application claims priority from U.S. provisional application 62/619,174, filed Jan. 19, 2018, which is incorporated herein by reference in its entirety.

### FIELD

The present application generally relates to pharmaceutical compositions comprising dasotraline.

### BACKGROUND

Development of a pharmaceutical composition provides for a plurality of considerations, such as route of administration (e.g. enteral, parenteral, topical, etc.), dosage form (solid—tablet, capsule, etc.; liquid—solution, suspension, syrup, etc.), strength of therapeutic component(s) (1-1,000 mg), non-therapeutic component(s) and their respective amounts, and each of these considerations require additional considerations such as stability, degradation, sensitivity to light, solubility, taste if administered enterally, palatability, pH, skin irritability, microbial growth, etc. Discovering and developing a pharmaceutical composition that addresses these considerations must also maintain the desired therapeutic effect. For example, certain patient populations may have difficulty ingesting pharmaceutical compositions in solid form (e.g. tablet, capsule, etc.), which may limit that population's treatment options if alternative dosage forms, such as a liquid dosage form, are not discovered, designed, clinically tested, and commercialized. Thus, in addressing an unmet need of treating populations with difficulty ingesting solid form pharmaceutical compositions (e.g. pediatrics, elderly subjects, etc.), a liquid form pharmaceutical composition for oral administration that provides a therapeutic effect while employing such considerations is desired.

### SUMMARY

Provided herein are pharmaceutical compositions for oral administration comprising dasotraline, or a pharmaceutically acceptable salt thereof, and one or more pharmaceutically acceptable excipients, wherein the pharmaceutical composition provides a decreased bitterness and/or paresthesic sensation.

In some embodiments, the one or more pharmaceutically acceptable excipients are each independently buffering agents, preservatives, or taste-masking agents.

In some embodiments, the pharmaceutical composition is chemically stable. In some embodiments, the pharmaceutical composition is resistant to microbial growth.

In some embodiments, the buffering agent is citric acid, tartaric acid, malic acid, acetic acid, lactic acid, phosphoric acid, maleic acid, sodium citrate, sodium tartrate, sodium malate, sodium acetate, sodium lactate or sodium dihydrogen phosphate dehydrate.

In some embodiments, the preservative is benzoic acid, sodium benzoate, methyl paraben, propyl paraben, propylene glycol, sorbic acid, potassium sorbate or sodium dehydroacetate.

In some embodiments, the taste-masking agent is cyclodextrin, or a derivative thereof. In some embodiments, the taste-masking agent is α-cyclodextrin, β-cyclodextrin, γ-cyclodextrin, hydroxypropyl-β-cyclodextrin, sulfobutylether-β-cyclodextrin sodium salt, randomly methylated β-cyclodextrin, branched β-cyclodextrins or hydroxypropyl-γ-cyclodextrins.

In some embodiments, the taste-masking agent is cyclodextrin, or a derivative thereof, and optionally a sweetening agent selected from the group consisting of aspartame, saccharin calcium, dextrose, fructose, maltitol, mannitol, saccharin, saccharin sodium, sorbitol, sucralose, sucrose, syrup, and acesulfame potassium

In some embodiments, provided are methods for decreasing bitterness or paresthesic sensation of a pharmaceutical composition comprising dasotraline, or a pharmaceutically acceptable salt thereof, by preparing a pharmaceutical composition comprising dasotraline, or a pharmaceutically acceptable salt thereof, and hydroxypropyl-β-cyclodextrin, and one or more pharmaceutically acceptable excipients.

In some embodiments, provided are methods for masking an undesirable taste or paresthesic sensation of a pharmaceutical composition comprising dasotraline, or a pharmaceutically acceptable salt thereof, by preparing a pharmaceutical composition comprising dasotraline, or a pharmaceutically acceptable salt thereof, and hydroxypropyl-β-cyclodextrin, and one or more pharmaceutically acceptable excipients.

In some embodiments, provided are methods for treating a disorder while decreasing bitterness or paresthesic sensation comprising administering a liquid pharmaceutical composition comprising dasotraline, or a pharmaceutically acceptable salt thereof, and one or more pharmaceutically acceptable excipients, wherein the administration comprises:

(a) measuring an amount of the pharmaceutical composition with a cup, a dosing spoon, or a dropper, or an oral syringe; and

(b) delivering the measured amount of the pharmaceutical composition to a patient in need thereof.

In some embodiments, provided is a flavoring system of a pharmaceutical composition comprising dasotraline, or a pharmaceutically acceptable salt thereof, wherein the pharmaceutical composition comprises one or more buffering agents, preservatives, or taste-masking agents.

### BRIEF DESCRIPTION OF THE FIGURES

The figures provided herein are made with the understanding that the present disclosure is to be considered as an exemplification of the claimed subject matter, and is not intended to limit the claims to the specific embodiments.

FIG. 1 is a chart displaying the results of the e-tongue test for dasotraline in water and Formulas F1, F2, F3, F4, F5, and F6 using BT0 and AN0 sensors.

FIG. 2 is a graph of bitterness intensity as a function of time for a 1 mg/mL formulation with 5% HPBC (solid line) and 2.5% HPBC (dashed line).

FIG. 3 is a graph of bitterness intensity as a function of time for a 0.5 mg/mL dasotraline formulation with 2.5% HPBC (solid line) and 1.25% HPBC (dashed line).

FIG. 4 is a graph of tongue sting and burning mouthfeel intensity as a function of time for a 1 mg/mL formulation with 5% HPBC (solid line) and 2.5% HPBC (dashed line).

FIG. 5 is a graph of tongue sting and burning mouthfeel intensity as a function of time for a 0.5 mg/mL dasotraline formulation with 2.5% HPBC (solid line) and 1.25% HPBC (dashed line).

FIG. 6 is a graph of cooling mouthfeel intensity as a function of time for a 1 mg/mL formulation with 5% HPBC (solid line) and 2.5% HPBC (dashed line).

FIG. 7 is a graph of cooling mouthfeel intensity as a function of time for a 0.5 mg/mL dasotraline formulation with 2.5% HPBC (solid line) and 1.25% HPBC (dashed line).

### DETAILED DESCRIPTION

The description provided herein is made with the understanding that the present disclosure is to be considered as an exemplification of the claimed subject matter and is not intended to limit the claims to the specific embodiments. The headings used throughout this disclosure are provided for convenience and are not to be construed to limit the claims in any way. Embodiments under any heading may be combined with embodiments under any other heading.

All published documents cited herein are hereby incorporated by reference in their entirety.

### Definitions

As used herein, the terms “comprising” and “including” or grammatical variants thereof are to be taken as specifying the stated features, integers, steps or components, but do not preclude the addition of one or more additional features, integers, steps, components or groups thereof. This term encompasses the terms “consisting of” and “consisting essentially of”. The phrase “consisting essentially of” or grammatical variants thereof, when used herein, are to be taken as specifying the stated features, integers, steps or components, but do not preclude the addition of one or more additional features, integers, steps, components or groups thereof, but only if the additional features, integers, steps, components or groups thereof do not materially alter the basic and novel characteristics of the claimed composition or method.

As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method that “comprises”, “has”, “includes” or “contains” one or more steps or elements possesses those one or more steps or elements, but is not limited to possessing only those one or more steps or elements.

As used herein, the term “subject,” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The “subject” may have independently been diagnosed with a disorder as defined herein, or may currently be experiencing symptoms associated with disorders, or may have experienced symptoms in the past, or may be at risk of developing a disorder, or may be reporting one or more of the symptoms of a disorder, even though a diagnosis may not have been made.

As used herein, the term “therapeutically effective amount” or “effective amount” refers to an amount that is effective to elicit the desired biological or medical response, including the amount of a compound that, when administered to a subject for treating a disorder, is sufficient to effect such treatment of the disorder. The effective amount will vary depending on the compound, the disorder, and its severity, and the age, weight, etc. of the patient to be treated. The effective amount may be in one or more doses (for example, a single dose or multiple doses may be required to achieve the desired treatment endpoint). An effective amount may be considered to be given in an effective amount if, in conjunction with one or more other agents, a desirable or beneficial result may be or is achieved. Suitable doses of any co-administered compounds may optionally be lowered due to the combined action, additive or synergistic, of the compound.

“Pharmaceutically acceptable” or “physiologically acceptable” refer to compounds, salts, hydrates, forms, compositions, dosage forms and other materials which are useful in preparing a pharmaceutical composition that is suitable for veterinary or human pharmaceutical use.

As used herein, the term “pharmaceutically acceptable excipient” includes, without limitation, any binder, buffering agent, filler, adjuvant, carrier, excipient, glidant, sweetening agent/sweetener, diluent, preservative, dye/colorant, flavor enhancer, surfactant, wetting agent, dispersing agent, suspending agent, thickening agent/thickener, stabilizer, antioxidant, isotonic agent, solvent, taste-masking agents, emulsifier, anti-caking agent, vehicle, flavoring agents/flavor, desiccants, plasticizers, disintegrants, or lubricant which has been approved by the United States Food and Drug Administration as being acceptable for use in humans or domestic animals.

Non-limiting examples of excipients are provided herein. Other excipients that are within the spirit of the invention are disclosed in the Handbook of Pharmaceutical Excipients, 8^{th} Edition, (Sheskey, Paul J. et al.) which is hereby incorporated by reference in its entirety. In certain embodiments, non-limiting examples of excipients include hydrochloric acid, sodium hydroxide, sodium dihydrogen phosphate, dibasic sodium phosphate, tribasic sodium phosphate, polysorbate 60, polysorbate 80, corn starch, potato starch, tapioca starch, or other starches, gelatin, natural and synthetic gums such as acacia, sodium alginate, alginic acid, other alginates, powdered tragacanth, guar gum, xanthan gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), talc, calcium carbonate (e.g., granules or powder), powdered cellulose, dextrates, kaolin, mannitol, silicic acid, sorbitol, starch, pre-gelatinized starch, agar-agar, alginic acid, calcium carbonate, cyclodextrin (α, β, α) or derivatives thereof, microcrystalline cellulose, croscarmellose sodium, crospovidone, polacrilin potassium, sodium starch glycolate, pre-gelatinized starch, other starches, clays, other algins, other celluloses, gums, calcium stearate, magnesium stearate, mineral oil, light mineral oil, glycerin, sorbitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, citric acid, benzoic acid, sodium benzoate, methyl paraben, propyl paraben, propylene glycol, sorbic acid, potassium sorbate, sodium dehydroacetate, tartaric acid, malic acid, acetic acid, lactic acid, phosphoric acid, maleic acid, sodium citrate, sodium tartrate, sodium malate, sodium acetate, sodium lactate, sodium dihydrogen phosphate dehydrate, acesulfame potassium, aspartame, saccharin calcium, dextrose, fructose, maltitol, saccharin, saccharin sodium, sorbitol, sucralose, sucrose, syrup, and acesulfame potassium and mixtures thereof.

As used herein, the terms “treatment” or “treating” are used interchangeably. These terms refer to an approach for obtaining beneficial or desired results including, but not limited to, therapeutic benefit. Therapeutic benefit includes eradication and/or amelioration of the underlying disorder being treated; it also includes the eradication and/or amelioration of one or more of the symptoms associated with the underlying disorder such that an improvement is observed in the patient, notwithstanding that the patient may still be afflicted with the underlying disorder. In some embodiments, “treatment” or “treating” includes one or more of the following: (a) inhibiting the disorder (for example, decreasing one or more symptoms resulting from the disorder, and/or diminishing the extent of the disorder); (b) slowing or arresting the development of one or more symptoms associated with the disorder (for example, stabilizing the disorder and/or delaying the worsening or progression of the disorder); and/or (c) relieving the disorder (for example, causing the regression of clinical symptoms, ameliorating the disorder, delaying the progression of the disorder, and/or increasing quality of life).

As used herein, the term “disorder,” or a specifically identified disorder disclosed herein, refers to the disorder as defined in the Diagnostic and Statistical Manual of Mental Disorders, Fifth Edition (DSM-5), which is incorporated by reference in its entirety. In some embodiments, a disorder is a Feeding and Eating Disorder as defined in the DSM-5. In some embodiments, the Feeding and Eating Disorder is pica, rumination disorder, avoidant/restrictive food intake disorder, anorexia nervosa, bulimia nervosa, binge eating disorder, other specified feeding or eating disorder, or unspecified feeding or eating disorder. In some embodiments, the Feeding and Eating Disorder is binge eating disorder. In some embodiments, a disorder is a Neurodevelopmental Disorder as defined in the DSM-5. In some embodiments, the Neurodevelopmental Disorder is an Intellectual Disability Disorder, Global Developmental Delay, Unspecified Intellectual Disability, Communication Disorder, Language Disorder, Speech Sound Disorder, Childhood-Onset Fluency Disorder (Stuttering), Social (Pragmatic) Communication Disorder, Unspecified Communication Disorder, Autism Spectrum Disorder, Attention-Deficit/Hyperactivity Disorder (ADHD), Other Specified Attention/Hyperactivity Disorder, Unspecified Attention-Deficit/Hyperactivity Disorder, Specific Learning Disorder, Motor Disorder, Developmental Coordination Disorder, Stereotypic Movement Disorder, Tic Disorder, Other Specified Tic Disorder, Unspecified Tic Disorder, Other Neurodevelopmental Disorders, Other Specified Neurodevelopmental Disorder, and Unspecified Neurodevelopmental Disorder. In some embodiments, the Neurodevelopmental Disorder is Attention-Deficit/Hyperactivity Disorder, Other Specified Attention/Hyperactivity Disorder, and Unspecified Attention-Deficit/Hyperactivity Disorder. In some embodiments, the Neurodevelopmental Disorder is Attention-Deficit/Hyperactivity Disorder.

As used herein, “delaying” development of a disorder mean to defer, hinder, slow, stabilize, and/or postpone development of the disorder. Delay can be of varying lengths of time, depending on the history of the disease and/or the individual being treated.

As used herein, “prevention” or “preventing” refers to a regimen that protects against the onset of the disorder such that the clinical symptoms of the disorder do not develop. Accordingly, “prevention” relates to administration of a therapy, including administration of a compound disclosed herein, to a patient before signs of the diseases are detectable in the patient (for example, administration of a compound disclosed herein to a patient in the absence of a detectable syndrome of the disorder). The patient may be an individual at risk for developing the disorder.

As used herein, an “at risk” individual is an individual who is at risk of developing a disorder to be treated. This may be shown, for example, by one or more risk factors, which are measurable parameters that correlate with development of a disorder and are known in the art.

As used herein “chemically stable” in reference to a pharmaceutical composition, describes a pharmaceutical composition that is resistant to decomposition when exposed to natural conditions, such as air, heat, light, pressure, or humidity for a period of time. In some embodiments, the period of time may be more than one week or more than two weeks or more than three weeks or more than four weeks or more than one month or more than two months or more than three months or more than four months or more than five months or more than six months. In some non-limiting examples, a chemically stable pharmaceutical composition is resistant to decomposition when exposed to air, heat, light, pressure, or humidity for more than one week or more than two weeks or more than three weeks or more than four weeks or more than one month or more than two months or more than three months or more than four months or more than five months or more than six months.

As used herein, “resistance to microbial growth” refers to the criteria or limits set by the United States Food and Drug Administration in the Pharmaceutical Microbiology Manual, which is hereby incorporated by reference in its entirety. In some non-limiting examples, a pharmaceutical composition is resistant to microbial growth if there is (bacteria) not less than 1.0 log reduction from the initial calculated count at 7 days, not less than 3.0 log reduction from the initial count at 14 days, and no increase from the 14 day count at 28 days and (yeast and molds) no increase from the initial calculated count at 7, 14, and 28 days. In some non-limiting examples, a pharmaceutical composition is resistant to microbial growth if there is (bacteria) not less than a 2.0 log reduction from the initial count at 14 days, and no increase from the 14 day count at 28 days and (yeast and molds) no increase from the initial calculated count at 14 and 28 days. In some non-limiting examples, a pharmaceutical composition is resistant to microbial growth if there is (bacteria) not less than a 1.0 log reduction from the initial count at 14 days, and no increase from the 14 day count at 28 days and (yeast and molds) no increase from the initial calculated count at 14 and 28 days. In some non-limiting examples, a pharmaceutical composition is resistant to microbial growth if there is (bacteria, yeast, and molds) no increase from the initial calculated count at 14 and 28 days.

As used herein, the term “bitter” or derivatives thereof (e.g. “bitterness”) refers to an undesirable taste perceived by a subject or measured using a sensor, such as an e-tongue sensor. As a non-limiting example, a substance may be considered bitter if the substance is perceived as unpleasant or sharp.

As used herein, the term “paresthesic” refers to a stinging or tingling or numbing sensation or feeling perceived by a subject.

As used herein, the term “independently” when used with a selection of excipients, means any one or none of the listed excipients may be included. As a non-limiting example, a pharmaceutical composition comprising dasotraline, or a pharmaceutically acceptable salt thereof, and one or more pharmaceutically acceptable excipients wherein the pharmaceutically acceptable excipients are independently buffering agent, preservative, or taste-masking agent, provides a pharmaceutical composition that may have one or more buffering agents and no preservatives or taste-masking agents; or a pharmaceutical composition that may have one or more buffering agents and one or more taste-masking agents and no preservatives; or a pharmaceutical composition having one or more buffering agents, and one or more taste-masking agents, and one or more preservatives; or a pharmaceutical composition comprising one of buffering agent, taste-masking agent, or preservative.

Compounds

The present disclosure provides the compound dasotraline:

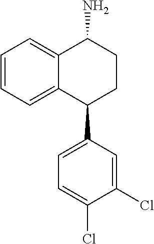 [SMILES: N[C@@H]1CC[C@@H](c2ccc(Cl)c(Cl)c2)c2ccccc21]

or a pharmaceutically acceptable salt thereof.

One of ordinary skill in the art would appreciate that nomenclature of compounds may vary. For example, dasotraline has an IUPAC name (1R,4S)-4-(3,4-dichlorophenyl)-1,2,3,4-tetrahydronaphthalen-1-amine. Dasotraline has a CAS registry number 675126-05-3.

Preparation of dasotraline can be found in PCT Publication No. WO2004/024669, for example, on page 6, example P, which is incorporated herein in its entirety.

Dasotraline is a novel compound with Norepinephrine Dopamine Reuptake Inhibitor (DNRI) pharmacology. Dasotraline acts as a potent inhibitor of human dopamine active transporters (DAT; dopamine uptake IC₅₀ 3 nM) and norepinephrine transporters (NET; norepinephrine uptake IC₅₀ 4 nM), and a weaker inhibitor of human serotonin transporters (SERT; serotonin uptake IC₅₀ 15 nM).

In a series of clinical trials dasotraline was administered via capsule at doses of 1 to 8 mg for an adult or adolescent and was effective in treating ADHD with no detectable abuse liability.

Dasotraline and dasotraline HCl have been found to have a naturally bitter taste and/or to provide a paresthesic feeling in the tongue, thus it is desirable, as provided herein, that in addressing an unmet need of treating populations with difficulty ingesting solid form pharmaceutical compositions (e.g. pediatrics, elderly subjects, etc.) that an orally administered pharmaceutical composition comprising dasotraline, or a pharmaceutically acceptable salt thereof, mask the taste and/or paresthesic sensation of dasotraline.

Amounts of dasotraline described herein, unless otherwise defined, are the amount calculated as the free base. The amounts can be adjusted according to the salt form of being employed. For example, 0.5625 mg of a hydrochloride salt of dasotraline is equivalent to 0.5 mg of the free base; or 1.125 mg of a hydrochloride salt of dasotraline is equivalent to 1.0 mg of the free base.

Provided are also pharmaceutically acceptable salts, hydrates, solvates, tautomeric forms, polymorphs, and prodrugs of dasotraline described herein. In some embodiments, provided is dasotraline HCl. In some embodiments, provided is anhydrous dasotraline HCl.

Dasotraline may be prepared and/or formulated as various pharmaceutically acceptable salts. For example, a pharmaceutically acceptable salt of dasotraline would include

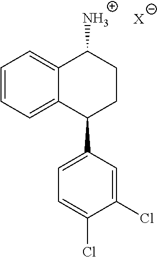

in which X⁻ is any counterion. In certain embodiments, X⁻ is the conjugate base of a pharmaceutically acceptable acid.

Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, .gamma.-hydroxybutyrates, glycolates, tartrates, and mandelates. Lists of other suitable pharmaceutically acceptable salts are found in Remington: The Science and Practice of Pharmacy, 21.sup.st Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds disclosed herein, can generally be prepared by conventional techniques known to those skilled in the art or by processes analogous to those described in the Examples as set out below using an appropriate isotopically-labeled reagent in place of the non-labeled reagent previously employed.

The compounds disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Likewise, all tautomeric forms are also intended to be included.

Excipients

Non-limiting examples of excipients are provided herein. Other excipients that are within the spirit of the invention are disclosed in the Handbook of Pharmaceutical Excipients, 8^{th} Edition, (Sheskey, Paul J. et al.) which is hereby incorporated by reference in its entirety.

Buffering Agents

Buffering agents can affect stability, pH and therapeutic effect of a pharmaceutical composition. Non-limiting examples of buffering agents include: citric acid, tartaric acid, malic acid, acetic acid, lactic acid, phosphoric acid, maleic acid, sodium citrate, sodium tartrate, sodium malate, sodium acetate, sodium lactate and sodium dihydrogen phosphate dihydrate.

In some embodiments, the buffering agent is citric acid. In some embodiments, the buffering agent is tartaric acid. In some embodiments, the buffering agent is malic acid. In some embodiments, the buffering agent is acetic acid.

In some embodiments, provided is a pharmaceutical composition comprising 5 mM to 50 mM (0.3 mg/mL to 9.6 mg/mL) of buffering agent. In some embodiments, provided is a pharmaceutical composition comprising 5 mM to 30 mM (0.3 mg/mL to 5.8 mg/mL) of buffering agent. In some embodiments, provided is a pharmaceutical composition comprising 10 mM±2.5 mM (0.6 mg/mL to 1.9 mg/mL) of buffering agent. In some embodiments, provided is a pharmaceutical composition comprising 25 mM±2.5 mM (1.5 mg/mL to 4.8 mg/mL) of buffering agent.

In some embodiments, provided is a pharmaceutical composition comprising 5 mM to 50 mM (0.7 mg/mL to 6.7 mg/mL) of malic acid. In some embodiments, provided is a pharmaceutical composition comprising 5 mM to 30 mM (0.7 mg/mL to 4.0 mg/mL) of malic acid. In some embodiments, provided is a pharmaceutical composition comprising 10 mM±2.5 mM (1.34±0.25 mg/mL) of malic acid. In some embodiments, provided is a pharmaceutical composition comprising 25 mM±2.5 mM (3.35±0.25 mg/mL) of malic acid.

In some embodiments, provided is a pharmaceutical composition comprising 0.3 mg/mL to 9.6 mg/mL (5 mM to 50 mM) of buffering agent. In some embodiments, provided is a pharmaceutical composition comprising 0.3 mg/mL to 5.8 mg/mL (5 mM to 30 mM) of buffering agent. In some embodiments, provided is a pharmaceutical composition comprising 0.6 mg/mL to 1.9 mg/mL (10 mM±2.5 mM) of buffering agent. In some embodiments, provided is a pharmaceutical composition comprising 1.5 mg/mL to 4.8 mg/mL (25 mM±2.5 mM) of buffering agent.

In some embodiments, provided is a pharmaceutical composition comprising 1.0 mg/mL to 9.6 mg/mL (5 mM to 50 mM) of citric acid. In some embodiments, provided is a pharmaceutical composition comprising 1.0 mg/mL to 5.8 mg/mL (5 mM to 30 mM) of citric acid. In some embodiments, provided is a pharmaceutical composition comprising 1.92±0.25 mg/mL (10 mM±2.5 mM) of citric acid. In some embodiments, provided is a pharmaceutical composition comprising 4.80±0.25 mg/mL (25 mM±2.5 mM) of citric acid.

In some embodiments, provided is a pharmaceutical composition comprising 0.8 mg/mL to 7.5 mg/mL (5 mM to 50 mM) of tartaric acid. In some embodiments, provided is a pharmaceutical composition comprising 0.8 mg/mL to 4.5 mg/mL (5 mM to 30 mM) of tartaric acid. In some embodiments, provided is a pharmaceutical composition comprising 1.50±0.25 mg/mL (10 mM±2.5 mM) of tartaric acid. In some embodiments, provided is a pharmaceutical composition comprising 3.75±0.25 mg/mL (25 mM±2.5 mM) of tartaric acid.

In some embodiments, provided is a pharmaceutical composition comprising 0.7 mg/mL to 6.7 mg/mL (5 mM to 50 mM) of malic acid. In some embodiments, provided is a pharmaceutical composition comprising 0.7 mg/mL to 4.0 mg/mL (5 mM to 30 mM) of malic acid. In some embodiments, provided is a pharmaceutical composition comprising 1.34±0.25 mg/mL (10 mM±2.5 mM) of malic acid. In some embodiments, provided is a pharmaceutical composition comprising 3.35±0.25 mg/mL (25 mM±2.5 mM) of malic acid.

In embodiments expressing concentration in one unit (i.e. mg/mL), other expressions of concentration are also included. In some embodiments, concentration of buffering agent is expressed as mg/mL. In some embodiments, concentration of buffering is expressed as mM.

Preservatives

Preservatives can affect stability, microbiological quality, pH and therapeutic effect. Non-limiting examples of preservatives include: benzoic acid, sodium benzoate, methyl paraben, propyl paraben, propylene glycol, sorbic acid, potassium sorbate and sodium dehydroacetate.

In some embodiments, the preservative is benzoic acid. In some embodiments, the preservative is sodium benzoate. In some embodiments, the preservative is methyl paraben. In some embodiments, the preservative is propyl paraben. In some embodiments, the preservative is propylene glycol.

In some embodiments, provided is a pharmaceutical composition comprising 1.0 mg/mL to 2.5 mg/mL of preservative. In some embodiments, provided is a pharmaceutical composition comprising 1.5 mg/mL to 2.5 mg/mL of preservative. In some embodiments, provided is a pharmaceutical composition comprising 2.0±0.25 mg/mL of preservative.

In some embodiments, provided is a pharmaceutical composition comprising 1.0 mg/mL to 2.5 mg/mL of benzoic acid. In some embodiments, provided is a pharmaceutical composition comprising 1.5 mg/mL to 2.5 mg/mL of benzoic acid. In some embodiments, provided is a pharmaceutical composition comprising 2.0±0.25 mg/mL of benzoic acid.

In some embodiments, provided is a pharmaceutical composition comprising 1.0 mg/mL to 2.5 mg/mL of sodium benzoate. In some embodiments, provided is a pharmaceutical composition comprising 1.5 mg/mL to 2.5 mg/mL of sodium benzoate. In some embodiments, provided is a pharmaceutical composition comprising 2.0±0.25 mg/mL of sodium benzoate.

In some embodiments, provided is a pharmaceutical composition comprising 1.0 mg/mL to 2.5 mg/mL of methyl paraben. In some embodiments, provided is a pharmaceutical composition comprising 1.5 mg/mL to 2.5 mg/mL of methyl paraben. In some embodiments, provided is a pharmaceutical composition comprising 2.0±0.25 mg/mL of methyl paraben.

In some embodiments, provided is a pharmaceutical composition comprising 1.0 mg/mL to 2.5 mg/mL of propyl paraben. In some embodiments, provided is a pharmaceutical composition comprising 1.5 mg/mL to 2.5 mg/mL of propyl paraben. In some embodiments, provided is a pharmaceutical composition comprising 2.0±0.25 mg/mL of propyl paraben.

Taste-Masking Agents

Taste-Masking Agents can affect taste, stability, palatability, pH, and therapeutic effect. Non-limiting examples of taste-masking agents include cyclodextrins and sweetening agents.

Non-limiting examples of cyclodextrins include α-cyclodextrins (six-membered sugar ring), β-cyclodextrins (seven-membered sugar ring), and γ-cyclodextrins (eight-membered sugar ring), and derivatives thereof. Non-limited examples of cyclodextrin derivatives include: methyl-β-cyclodextrin, hydroxypropyl-β-cyclodextrin, sulfobutylether-β-cyclodextrin sodium salt, randomly methylated β-cyclodextrin, branched β-cyclodextrins and hydroxypropyl-γ-cyclodextrins.

In some embodiments, the cyclodextrin or derivative thereof is an α-cyclodextrin or derivative thereof. In some embodiments, the cyclodextrin or derivative thereof is a β-cyclodextrin or derivative thereof. In some embodiments, the cyclodextrin or derivative thereof is a γ-cyclodextrin or derivative thereof. In some embodiments, the cyclodextrin or derivative thereof is methyl-β-cyclodextrin. In some embodiments, the cyclodextrin or derivative thereof is hydroxypropyl-β-cyclodextrin. β-cyclodextrin can be abbreviated at “βCD” as used herein. Hydroxypropyl-β-cyclodextrin can be abbreviated at “HPBC” as used herein. Hydroxypropyl-β-cyclodextrin is also known as hydroxypropyl betadex.

In some embodiments, provided is a pharmaceutical composition comprising 10 mg/mL to 60 mg/mL of cyclodextrin or derivative thereof. In some embodiments, provided is a pharmaceutical composition comprising 10 mg/mL to 30 mg/mL of cyclodextrin or derivative thereof. In some embodiments, provided is a pharmaceutical composition comprising 20 mg/mL to 55 mg/mL of cyclodextrin or derivative thereof. In some embodiments, provided is a pharmaceutical composition comprising 12±2.5 mg/mL of cyclodextrin or derivative thereof. In some embodiments, provided is a pharmaceutical composition comprising 25±5 mg/mL of cyclodextrin or derivative thereof. In some embodiments, provided is a pharmaceutical composition comprising 50±5 mg/mL of cyclodextrin or derivative thereof.

In some embodiments, provided is a pharmaceutical composition comprising 10 mg/mL to 60 mg/mL of β-cyclodextrin or derivative thereof. In some embodiments, provided is a pharmaceutical composition comprising 10 mg/mL to 30 mg/mL of β-cyclodextrin or derivative thereof. In some embodiments, provided is a pharmaceutical composition comprising 20 mg/mL to 55 mg/mL of β-cyclodextrin or derivative thereof. In some embodiments, provided is a pharmaceutical composition comprising 12±2.5 mg/mL of β-cyclodextrin or derivative thereof. In some embodiments, provided is a pharmaceutical composition comprising 25±5 mg/mL of β-cyclodextrin or derivative thereof. In some embodiments, provided is a pharmaceutical composition comprising 50±5 mg/mL of β-cyclodextrin or derivative thereof.

In some embodiments, provided is a pharmaceutical composition comprising 10 mg/mL to 60 mg/mL of methyl-β-cyclodextrin. In some embodiments, provided is a pharmaceutical composition comprising 10 mg/mL to 30 mg/mL of methyl-β-cyclodextrin. In some embodiments, provided is a pharmaceutical composition comprising 20 mg/mL to 55 mg/mL of methyl-β-cyclodextrin. In some embodiments, provided is a pharmaceutical composition comprising 12±2.5 mg/mL of methyl-β-cyclodextrin. In some embodiments, provided is a pharmaceutical composition comprising 25±5 mg/mL of methyl-β-cyclodextrin. In some embodiments, provided is a pharmaceutical composition comprising 50±5 mg/mL of methyl-β-cyclodextrin.

In some embodiments, provided is a pharmaceutical composition comprising 10 mg/mL to 60 mg/mL of hydroxypropyl-β-cyclodextrin. In some embodiments, provided is a pharmaceutical composition comprising 10 mg/mL to 30 mg/mL of hydroxypropyl-β-cyclodextrin. In some embodiments, provided is a pharmaceutical composition comprising 20 mg/mL to 55 mg/mL of hydroxypropyl-3-cyclodextrin. In some embodiments, provided is a pharmaceutical composition comprising 12±2.5 mg/mL of hydroxypropyl-3-cyclodextrin. In some embodiments, provided is a pharmaceutical composition comprising 25±5 mg/mL of hydroxypropyl-β-cyclodextrin. In some embodiments, provided is a pharmaceutical composition comprising 50±5 mg/mL of hydroxypropyl-3-cyclodextrin.

Non-limiting examples of sweetening agents include: aspartame, saccharin calcium, dextrose, fructose, maltitol, mannitol, saccharin, saccharin sodium, sorbitol, sucralose, sucrose, syrup, and acesulfame potassium.

In some embodiments, the sweetening agent is sucralose.

In some embodiments, provided is a pharmaceutical composition comprising 0.05 mg/mL to 0.2 mg/mL of sweetening agent. In some embodiments, provided is a pharmaceutical composition comprising 0.05 mg/mL to 0.15 mg/mL of sweetening agent. In some embodiments, provided is a pharmaceutical composition comprising 0.1±0.05 mg/mL of sweetening agent.

In some embodiments, provided is a pharmaceutical composition comprising 0.05 mg/mL to 0.2 mg/mL of sucralose. In some embodiments, provided is a pharmaceutical composition comprising 0.05 mg/mL to 0.15 mg/mL of sucralose. In some embodiments, provided is a pharmaceutical composition comprising 0.1±0.05 mg/mL of sucralose.

Compositions

In some embodiments, provided are pharmaceutical composition comprising dasotraline, or a pharmaceutically acceptable salt thereof, and one or more pharmaceutically acceptable excipients.

In some embodiments, provided is a pharmaceutical composition comprising dasotraline, or a pharmaceutically acceptable salt thereof, and one or more pharmaceutically acceptable excipients wherein the pharmaceutically acceptable excipients are independently buffering agents, preservatives, or taste-masking agents. In some embodiments, provided is a pharmaceutical composition comprising dasotraline, or a pharmaceutically acceptable salt thereof, and one to five pharmaceutically acceptable excipients wherein the pharmaceutically acceptable excipients are independently buffering agents, preservatives, or taste masking agents. In some embodiments, provided is a pharmaceutical composition comprising dasotraline, or a pharmaceutically acceptable salt thereof, and two pharmaceutically acceptable excipients wherein the pharmaceutically acceptable excipients are independently buffering agents, preservatives, or taste-masking agents. In some embodiments, provided is a pharmaceutical composition comprising dasotraline, or a pharmaceutically acceptable salt thereof, and three pharmaceutically acceptable excipients wherein the pharmaceutically acceptable excipients are independently buffering agents, preservatives, or taste-masking agents. In some embodiments, provided is a pharmaceutical composition comprising dasotraline, or a pharmaceutically acceptable salt thereof, and four pharmaceutically acceptable excipients wherein the pharmaceutically acceptable excipients are independently buffering agents, preservatives, or taste-masking agents. In some embodiments, provided is a pharmaceutical composition comprising dasotraline, or a pharmaceutically acceptable salt thereof, and five pharmaceutically acceptable excipients wherein the pharmaceutically acceptable excipients are independently buffering agents, preservatives, or taste-masking agents.

In some embodiments, provided is a pharmaceutical composition comprising dasotraline, or a pharmaceutically acceptable salt thereof, a buffering agent, and a cyclodextrin or derivative thereof. In some embodiments, provided is a pharmaceutical composition comprising dasotraline, or a pharmaceutically acceptable salt thereof, a buffering agent, a cyclodextrin or derivative thereof, and optionally a preservative. In some embodiments, provided is a pharmaceutical composition comprising dasotraline, or a pharmaceutically acceptable salt thereof, a buffering agent, a cyclodextrin or derivative thereof, and optionally a sweetening agent. In some embodiments, provided is a pharmaceutical composition comprising dasotraline, or a pharmaceutically acceptable salt thereof, a buffering agent, a cyclodextrin or derivative thereof, and optionally a preservative or a sweetening agent. In some embodiments, provided is a pharmaceutical composition comprising dasotraline, or a pharmaceutically acceptable salt thereof, a buffering agent, a cyclodextrin or derivative thereof, and optionally a preservative and a sweetening agent.

In some embodiments, provided is a pharmaceutical composition comprising dasotraline, or a pharmaceutically acceptable salt thereof, a buffering agent, a preservative, a cyclodextrin or derivative thereof, and a sweetening agent.

In some embodiments, provided is a pharmaceutical composition for oral administration comprising dasotraline, or a pharmaceutically acceptable salt thereof, a buffering agent, a preservative, a cyclodextrin or derivative thereof, and a sweetening agent.

In some embodiments, the pharmaceutical composition is a liquid for oral administration. In some embodiments, the pharmaceutical composition is a syrup, an oral suspension, an oral solution, an oral drop, an oral emulsion, a linctuse, or an elixir. In some embodiments, the pharmaceutical composition is an oral solution. In some embodiments, the pharmaceutical composition is an oral drop.

In some embodiments, the pharmaceutical composition is a liquid wherein the pharmaceutical composition is accompanied by a measuring device. In some embodiments, the pharmaceutical composition is an oral drop wherein the pharmaceutical composition is accompanied by a measuring device. In some embodiments, the pharmaceutical composition is an oral solution wherein the pharmaceutical composition is accompanied by a measuring device. In some embodiments, the measuring device is a cup, a dosing spoon, a dropper, or an oral syringe, or combination thereof. In some embodiments, the measuring device is a cup. In some embodiments, the measuring device is a dosing spoon. In some embodiments, the measuring device is a dropper. In some embodiments, the measuring device is an oral syringe.

In some embodiments, provided is a pharmaceutical composition for oral administration comprising dasotraline, or a pharmaceutically acceptable salt thereof, malic acid, and a cyclodextrin or derivative thereof. In some embodiments, provided is a pharmaceutical composition for oral administration comprising dasotraline, or a pharmaceutically acceptable salt thereof, malic acid, and a cyclodextrin or derivative thereof, and optionally a preservative. In some embodiments, provided is a pharmaceutical composition for oral administration comprising dasotraline, or a pharmaceutically acceptable salt thereof, malic acid, and a cyclodextrin or derivative thereof, and optionally a sweetening agent. In some embodiments, provided is a pharmaceutical composition for oral administration comprising dasotraline, or a pharmaceutically acceptable salt thereof, malic acid, and a cyclodextrin or derivative thereof, and optionally a preservative or a sweetening agent. In some embodiments, provided is a pharmaceutical composition for oral administration comprising dasotraline, or a pharmaceutically acceptable salt thereof, malic acid, and a cyclodextrin or derivative thereof, and optionally a preservative and a sweetening agent. In some embodiments, provided is a pharmaceutical composition for oral administration comprising dasotraline, or a pharmaceutically acceptable salt thereof, malic acid, and a cyclodextrin or derivative thereof, and optionally benzoic acid and sucralose. In some embodiments, provided is a pharmaceutical composition for oral administration comprising dasotraline, or a pharmaceutically acceptable salt thereof, malic acid, and a cyclodextrin or derivative thereof, and optionally benzoic acid and sucralose.

In some embodiments, provided is a pharmaceutical composition for oral administration comprising dasotraline, or a pharmaceutically acceptable salt thereof, a buffering agent, and hydroxypropyl-β-cyclodextrin. In some embodiments, provided is a pharmaceutical composition for oral administration comprising dasotraline, or a pharmaceutically acceptable salt thereof, a buffering agent, and hydroxypropyl-β-cyclodextrin, and optionally a preservative. In some embodiments, provided is a pharmaceutical composition for oral administration comprising dasotraline, or a pharmaceutically acceptable salt thereof, a buffering agent, and hydroxypropyl-β-cyclodextrin, and optionally a sweetening agent. In some embodiments, provided is a pharmaceutical composition for oral administration comprising dasotraline, or a pharmaceutically acceptable salt thereof, a buffering agent, and hydroxypropyl-β-cyclodextrin, and optionally a preservative or a sweetening agent. In some embodiments, provided is a pharmaceutical composition for oral administration comprising dasotraline, or a pharmaceutically acceptable salt thereof, a buffering agent, and hydroxypropyl-β-cyclodextrin, and optionally a preservative and a sweetening agent. In some embodiments, provided is a pharmaceutical composition for oral administration comprising dasotraline, or a pharmaceutically acceptable salt thereof, a buffering agent, and hydroxypropyl-β-cyclodextrin, and optionally benzoic acid and sucralose.

In some embodiments, provided is a pharmaceutical composition for oral administration comprising dasotraline, or a pharmaceutically acceptable salt thereof, malic acid, and hydroxypropyl-β-cyclodextrin. In some embodiments, provided is a pharmaceutical composition for oral administration comprising dasotraline, or a pharmaceutically acceptable salt thereof, malic acid, and hydroxypropyl-β-cyclodextrin, and optionally a preservative. In some embodiments, provided is a pharmaceutical composition for oral administration comprising dasotraline, or a pharmaceutically acceptable salt thereof, malic acid, and hydroxypropyl-β-cyclodextrin, and optionally a sweetening agent. In some embodiments, provided is a pharmaceutical composition for oral administration comprising dasotraline, or a pharmaceutically acceptable salt thereof, malic acid, and hydroxypropyl-β-cyclodextrin, and optionally a preservative or a sweetening agent. In some embodiments, provided is a pharmaceutical composition for oral administration comprising dasotraline, or a pharmaceutically acceptable salt thereof, malic acid, and hydroxypropyl-β-cyclodextrin, and optionally a preservative and a sweetening agent. In some embodiments, provided is a pharmaceutical composition for oral administration comprising dasotraline, or a pharmaceutically acceptable salt thereof, malic acid, and hydroxypropyl-β-cyclodextrin, and optionally benzoic acid and sucralose.

In some embodiments, provided is a pharmaceutical composition for oral administration comprising dasotraline, or a pharmaceutically acceptable salt thereof, malic acid, a preservative, a cyclodextrin or derivative thereof, and a sweetening agent. In some embodiments, provided is a pharmaceutical composition for oral administration comprising dasotraline, or a pharmaceutically acceptable salt thereof, a buffering agent, benzoic acid, a cyclodextrin or derivative thereof, and a sweetening agent. In some embodiments, provided is a pharmaceutical composition for oral administration comprising dasotraline, or a pharmaceutically acceptable salt thereof, a buffering agent, a preservative, hydroxypropyl-β-cyclodextrin, and a sweetening agent. In some embodiments, provided is a pharmaceutical composition for oral administration comprising dasotraline, or a pharmaceutically acceptable salt thereof, a buffering agent, a preservative, a cyclodextrin or derivative thereof, and sucralose.

In some embodiments, provided is a pharmaceutical composition for oral administration comprising a unit dose of 0.5 mg to 8 mg dasotraline, or a pharmaceutically acceptable salt thereof, a buffering agent, a preservative, a cyclodextrin or derivative thereof, and a sweetening agent. In some embodiments, provided is a pharmaceutical composition for oral administration comprising a unit dose of 1 mg to 8 mg dasotraline, or a pharmaceutically acceptable salt thereof, a buffering agent, a preservative, a cyclodextrin or derivative thereof, and a sweetening agent. In some embodiments, provided is a pharmaceutical composition for oral administration comprising a unit dose of 1 mg to 8 mg dasotraline, or a pharmaceutically acceptable salt thereof, a buffering agent, a preservative, a cyclodextrin or derivative thereof, and a sweetening agent, in aqueous solution at pH at 2.0 to 3.5. In some embodiments, provided is a pharmaceutical composition for oral administration comprising a unit dose of 1 mg to 8 mg dasotraline, or a pharmaceutically acceptable salt thereof, a buffering agent, a preservative, a cyclodextrin or derivative thereof, and a sweetening agent, in aqueous solution at pH at 2.6 to 3.2. In some embodiments, provided is a pharmaceutical composition for oral administration comprising a unit dose of 1 mg to 8 mg dasotraline, or a pharmaceutically acceptable salt thereof, a buffering agent, a preservative, a cyclodextrin or derivative thereof, and a sweetening agent, in aqueous solution at pH at 2.2 to 2.8.

In some embodiments, provided is a pharmaceutical composition for oral administration comprising a unit dose of 0.5 mg, 1.0 mg, 2.0 mg, 3.0 mg, 4.0 mg, 5.0 mg, 6.0 mg, 7.0 mg or 8 mg dasotraline, or a pharmaceutically acceptable salt thereof, a buffering agent, and a cyclodextrin or derivative thereof. In some embodiments, the unit dose is packaged as a cup, a dosing spoon, a dropper, or an oral syringe.

In some embodiments, provided is a pharmaceutical composition for oral administration comprising 0.4 mg/mL to 1.2 mg/mL dasotraline, or a pharmaceutically acceptable salt thereof, a buffering agent, a preservative, a cyclodextrin or derivative thereof, and a sweetening agent. In some embodiments, provided is a pharmaceutical composition for oral administration comprising 0.4 mg/mL to 1.2 mg/mL dasotraline, or a pharmaceutically acceptable salt thereof, a buffering agent, a preservative, a cyclodextrin or derivative thereof, and a sweetening agent, in aqueous solution at pH at 2.0 to 3.5. In some embodiments, provided is a pharmaceutical composition for oral administration comprising 0.4 mg/mL to 1.2 mg/mL dasotraline, or a pharmaceutically acceptable salt thereof, a buffering agent, a preservative, a cyclodextrin or derivative thereof, and a sweetening agent, in aqueous solution at pH at 2.6 to 3.2. In some embodiments, provided is a pharmaceutical composition for oral administration comprising 0.4 mg/mL to 1.2 mg/mL dasotraline, or a pharmaceutically acceptable salt thereof, a buffering agent, a preservative, a cyclodextrin or derivative thereof, and a sweetening agent, in aqueous solution at pH at 2.2 to 2.8.

In some embodiments, provided is a pharmaceutical composition comprising

a) 0.4 mg/mL to 1.2 mg/mL dasotraline, or a pharmaceutically acceptable salt thereof;

b) 0.3 mg/mL to 9.6 mg/mL (5 mM to 50 mM) buffering agent;

c) optionally 1 mg/mL to 2.5 mg/mL preservative;

d) 10 mg/mL to 60 mg/mL cyclodextrin or derivative thereof; and

e) optionally 0.05 mg/mL to 0.2 mg/mL sweetening agent.

In some embodiments, provided is a pharmaceutical composition for oral administration comprising

a) 0.4 mg/mL to 1.2 mg/mL dasotraline, or a pharmaceutically acceptable salt thereof;

b) 0.3 mg/mL to 9.6 mg/mL (5 mM to 50 mM) buffering agent;

c) optionally 1 mg/mL to 2.5 mg/mL preservative;

d) 10 mg/mL to 60 mg/mL cyclodextrin or derivative thereof; and

e) optionally 0.05 mg/mL to 0.2 mg/mL sweetening agent.

In some embodiments, provided is a pharmaceutical composition for oral administration comprising

a) 0.4 mg/mL to 1.2 mg/mL dasotraline, or a pharmaceutically acceptable salt thereof;

b) 0.3 mg/mL to 9.6 mg/mL (5 mM to 50 mM) buffering agent;

c) optionally 1 mg/mL to 2.5 mg/mL preservative;

d) 10 mg/mL to 60 mg/mL cyclodextrin or derivative thereof; and

e) optionally 0.05 mg/mL to 0.2 mg/mL sweetening agent in aqueous solution at pH 2.0 to 3.5.

In some embodiments, provided is a pharmaceutical composition for oral administration comprising

a) 0.4 mg/mL to 0.6 mg/mL dasotraline, or a pharmaceutically acceptable salt thereof;

b) 0.3 mg/mL to 9.6 mg/mL (5 mM to 50 mM) buffering agent;

c) optionally 1 mg/mL to 2.5 mg/mL preservative;

d) 10 mg/mL to 30 mg/mL cyclodextrin or derivative thereof; and

e) optionally 0.05 mg/mL to 0.2 mg/mL sweetening agent.

In some embodiments, provided is a pharmaceutical composition for oral administration comprising

a) 0.4 mg/mL to 0.6 mg/mL dasotraline, or a pharmaceutically acceptable salt thereof;

b) 0.3 mg/mL to 5.8 mg/mL (5 mM to 30 mM) buffering agent;

c) optionally 1.5 mg/mL to 2.5 mg/mL preservative;

d) 10 mg/mL to 30 mg/mL cyclodextrin or derivative thereof; and

e) optionally 0.05 mg/mL to 0.15 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 0.6 mg/mL to 1.9 mg/mL (10 mM) buffering agent;

c) optionally 2.0 mg/mL±0.25 mg/mL preservative;

d) 25 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 1.5 mg/mL to 4.8 mg/mL (25 mM) buffering agent;

c) optionally 2.0 mg/mL±0.25 mg/mL preservative;

d) 12.5 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 10 mM±2.5 mM buffering agent;

c) optionally 2.0 mg/mL±0.25 mg/mL preservative;

d) 25 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 25 mM±2.5 mM buffering agent;

c) optionally 2.0 mg/mL±0.25 mg/mL preservative;

d) 12.5 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 0.9 mg/mL to 1.1 mg/mL dasotraline, or a pharmaceutically acceptable salt thereof;

b) 0.3 mg/mL to 9.6 mg/mL (5 mM to 50 mM) buffering agent;

c) optionally 1 mg/mL to 2.5 mg/mL preservative;

d) 20 mg/mL to 55 mg/mL cyclodextrin or derivative thereof; and

e) optionally 0.05 mg/mL to 0.2 mg/mL sweetening agent.

In some embodiments, provided is a pharmaceutical composition for oral administration comprising

a) 0.9 mg/mL to 1.1 mg/mL dasotraline, or a pharmaceutically acceptable salt thereof;

b) 0.3 mg/mL to 5.8 mg/mL (5 mM to 30 mM) buffering agent;

c) optionally 1.5 mg/mL to 2.5 mg/mL preservative;

d) 20 mg/mL to 55 mg/mL cyclodextrin or derivative thereof; and

e) optionally 0.05 mg/mL to 0.15 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 0.6 mg/mL to 1.9 mg/mL (10 mM) buffering agent;

c) optionally 2.0 mg/mL±0.25 mg/mL preservative;

d) 50 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 1.5 mg/mL to 4.8 mg/mL (25 mM) buffering agent;

c) optionally 2.0 mg/mL±0.25 mg/mL preservative;

d) 25.0 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 10 mM±2.5 mM buffering agent;

c) optionally 2.0 mg/mL±0.25 mg/mL preservative;

d) 50 mg/mL±5.0 mg/mL cyclodextrin or derivative thereof; and

e) optionally 0.1 mg/mL±0.05 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 25 mM±2.5 mM buffering agent;

c) optionally 2.0 mg/mL±0.25 mg/mL preservative;

d) 25.0 mg/mL±5.0 mg/mL cyclodextrin or derivative thereof; and

e) optionally 0.1 mg/mL±0.05 mg/mL sweetening agent.

In some embodiments, provided is a pharmaceutical composition comprising

a) 0.4 mg/mL to 1.2 mg/mL dasotraline, or a pharmaceutically acceptable salt thereof;

b) 0.7 mg/mL to 6.7 mg/mL (5 mM to 50 mM) malic acid;

c) optionally 1 mg/mL to 2.5 mg/mL benzoic acid;

d) 10 mg/mL to 60 mg/mL hydroxypropyl-β-cyclodextrin; and

e) optionally 0.05 mg/mL to 0.2 mg/mL sucralose.

In some embodiments, provided is a pharmaceutical composition for oral administration comprising

a) 0.4 mg/mL to 1.2 mg/mL dasotraline, or a pharmaceutically acceptable salt thereof;

b) 0.7 mg/mL to 6.7 mg/mL (5 mM to 50 mM) malic acid;

c) optionally 1 mg/mL to 2.5 mg/mL benzoic acid;

d) 10 mg/mL to 60 mg/mL hydroxypropyl-β-cyclodextrin; and

e) optionally 0.05 mg/mL to 0.2 mg/mL sucralose.

In some embodiments, provided is a pharmaceutical composition for oral administration comprising

a) 0.4 mg/mL to 1.2 mg/mL dasotraline, or a pharmaceutically acceptable salt thereof;

b) 0.7 mg/mL to 6.7 mg/mL (5 mM to 50 mM) malic acid;

c) optionally 1 mg/mL to 2.5 mg/mL benzoic acid;

d) 10 mg/mL to 60 mg/mL hydroxypropyl-β-cyclodextrin; and

e) optionally 0.05 mg/mL to 0.2 mg/mL sucralose in aqueous solution at pH 2.0 to 3.5.

In some embodiments, provided is a pharmaceutical composition for oral administration comprising

a) 0.4 mg/mL to 0.6 mg/mL dasotraline, or a pharmaceutically acceptable salt thereof;

b) 0.7 mg/mL to 6.7 mg/mL (5 mM to 50 mM) malic acid;

c) optionally 1 mg/mL to 2.5 mg/mL benzoic acid;

d) 10 mg/mL to 30 mg/mL hydroxypropyl-β-cyclodextrin; and

e) optionally 0.05 mg/mL to 0.2 mg/mL sucralose.

In some embodiments, provided is a pharmaceutical composition for oral administration comprising

a) 0.4 mg/mL to 0.6 mg/mL dasotraline, or a pharmaceutically acceptable salt thereof;

b) 0.7 mg/mL to 4.0 mg/mL (5 mM to 30 mM) malic acid;

c) optionally 1.5 mg/mL to 2.5 mg/mL benzoic acid;

d) 10 mg/mL to 30 mg/mL hydroxypropyl-β-cyclodextrin; and

e) optionally 0.05 mg/mL to 0.15 mg/mL sucralose.

In some embodiments, provided is a pharmaceutical composition for oral administration comprising

a) 0.5 mg/mL±0.1 mg/mL dasotraline, or a pharmaceutically acceptable salt thereof;

b) 1.34 mg/mL±0.25 mg/mL (10 mM±2.5 mM) malic acid;

c) optionally 2.0 mg/mL±0.25 mg/mL benzoic acid;

d) 25 mg/mL±5.0 mg/mL hydroxypropyl-3-cyclodextrin; and

e) optionally 0.1 mg/mL±0.05 mg/mL sucralose.

In some embodiments, provided is a pharmaceutical composition for oral administration comprising

a) 0.5 mg/mL±0.1 mg/mL dasotraline, or a pharmaceutically acceptable salt thereof;

b) 1.34 mg/mL±0.25 mg/mL (10 mM±2.5 mM) malic acid;

c) optionally 2.0 mg/mL±0.25 mg/mL benzoic acid;

d) 25 mg/mL±5.0 mg/mL hydroxypropyl-3-cyclodextrin; and

e) optionally 0.1 mg/mL±0.05 mg/mL sucralose in aqueous solution at pH 2.6 to 3.2.

In some embodiments, provided is a pharmaceutical composition for oral administration comprising

a) 0.5 mg/mL±0.1 mg/mL dasotraline, or a pharmaceutically acceptable salt thereof;

b) 3.35 mg/mL±0.25 mg/mL (25 mM±2.5 mM) malic acid;

c) optionally 2.0 mg/mL±0.25 mg/mL benzoic acid;

d) 12.5 mg/mL±5.0 mg/mL hydroxypropyl-β-cyclodextrin; and

e) optionally 0.1 mg/mL±0.05 mg/mL sucralose.

In some embodiments, provided is a pharmaceutical composition for oral administration comprising

a) 0.5 mg/mL±0.1 mg/mL dasotraline, or a pharmaceutically acceptable salt thereof;

b) 3.35 mg/mL±0.25 mg/mL (25 mM±2.5 mM) malic acid;

c) optionally 2.0 mg/mL±0.25 mg/mL benzoic acid;

d) 12.5 mg/mL±5.0 mg/mL hydroxypropyl-β-cyclodextrin; and

e) optionally 0.1 mg/mL±0.05 mg/mL sucralose in aqueous solution at pH 2.2 to 2.8.

In some embodiments, provided is a pharmaceutical composition for oral administration comprising

a) 0.9 mg/mL to 1.1 mg/mL dasotraline, or a pharmaceutically acceptable salt thereof;

b) 0.7 mg/mL to 6.7 mg/mL (5 mM to 50 mM) malic acid;

c) optionally 1 mg/mL to 2.5 mg/mL benzoic acid;

d) 20 mg/mL to 55 mg/mL hydroxypropyl-β-cyclodextrin; and

e) optionally 0.05 mg/mL to 0.2 mg/mL sucralose.

In some embodiments, provided is a pharmaceutical composition for oral administration comprising

a) 0.9 mg/mL to 1.1 mg/mL dasotraline, or a pharmaceutically acceptable salt thereof;

b) 0.7 mg/mL to 4.0 mg/mL (5 mM to 30 mM) malic acid;

c) optionally 1.5 mg/mL to 2.5 mg/mL benzoic acid;

d) 20 mg/mL to 55 mg/mL hydroxypropyl-β-cyclodextrin; and

e) optionally 0.05 mg/mL to 0.15 mg/mL sucralose.

In some embodiments, provided is a pharmaceutical composition for oral administration comprising

a) 1.0 mg/mL±0.1 mg/mL dasotraline, or a pharmaceutically acceptable salt thereof;

b) 1.34 mg/mL±0.25 mg/mL (10 mM±2.5 mM) malic acid;

c) optionally 2.0 mg/mL±0.25 mg/mL benzoic acid;

d) 50 mg/mL±5.0 mg/mL hydroxypropyl-3-cyclodextrin; and

e) optionally 0.1 mg/mL±0.05 mg/mL sucralose.

In some embodiments, provided is a pharmaceutical composition for oral administration comprising

a) 1.0 mg/mL±0.1 mg/mL dasotraline, or a pharmaceutically acceptable salt thereof;

b) 1.34 mg/mL±0.25 mg/mL (10 mM±2.5 mM) malic acid;

c) optionally 2.0 mg/mL±0.25 mg/mL benzoic acid;

d) 50 mg/mL±5.0 mg/mL hydroxypropyl-3-cyclodextrin; and

e) optionally 0.1 mg/mL±0.05 mg/mL sucralose in aqueous solution at pH 2.6 to 3.2.

In some embodiments, provided is a pharmaceutical composition for oral administration comprising

a) 1.0 mg/mL±0.1 mg/mL dasotraline, or a pharmaceutically acceptable salt thereof;

b) 3.35 mg/mL±0.25 mg/mL (25 mM±2.5 mM) malic acid;

c) optionally 2.0 mg/mL±0.25 mg/mL benzoic acid;

d) 25.0 mg/mL±5.0 mg/mL hydroxypropyl-β-cyclodextrin; and

e) optionally 0.1 mg/mL±0.05 mg/mL sucralose.

In some embodiments, provided is a pharmaceutical composition for oral administration comprising

a) 1.0 mg/mL±0.1 mg/mL dasotraline, or a pharmaceutically acceptable salt thereof;

b) 3.35 mg/mL±0.25 mg/mL (25 mM±2.5 mM) malic acid;

c) optionally 2.0 mg/mL±0.25 mg/mL benzoic acid;

d) 25.0 mg/mL±5.0 mg/mL hydroxypropyl-β-cyclodextrin; and

e) optionally 0.1 mg/mL±0.05 mg/mL sucralose in aqueous solution at pH 2.2 to 2.8.

In some embodiments, provided is a pharmaceutical composition comprising

a) 0.4 mg/mL to 1.2 mg/mL dasotraline, or a pharmaceutically acceptable salt thereof;

b) 0.3 mg/mL to 9.6 mg/mL (5 mM to 50 mM) buffering agent;

c) 1 mg/mL to 2.5 mg/mL preservative;

d) 10 mg/mL to 60 mg/mL cyclodextrin or derivative thereof; and

e) 0.05 mg/mL to 0.2 mg/mL sweetening agent.

In some embodiments, provided is a pharmaceutical composition for oral administration comprising

a) 0.4 mg/mL to 1.2 mg/mL dasotraline, or a pharmaceutically acceptable salt thereof;

b) 0.3 mg/mL to 9.6 mg/mL (5 mM to 50 mM) buffering agent;

c) 1 mg/mL to 2.5 mg/mL preservative;

d) 10 mg/mL to 60 mg/mL cyclodextrin or derivative thereof; and

e) 0.05 mg/mL to 0.2 mg/mL sweetening agent.

In some embodiments, provided is a pharmaceutical composition for oral administration comprising

a) 0.4 mg/mL to 1.2 mg/mL dasotraline, or a pharmaceutically acceptable salt thereof;

b) 0.3 mg/mL to 9.6 mg/mL (5 mM to 50 mM) buffering agent;

c) 1 mg/mL to 2.5 mg/mL preservative;

d) 10 mg/mL to 60 mg/mL cyclodextrin or derivative thereof; and

e) 0.05 mg/mL to 0.2 mg/mL sweetening agent in aqueous solution at pH 2.0 to 3.5.

In some embodiments, provided is a pharmaceutical composition for oral administration comprising

a) 0.4 mg/mL to 0.6 mg/mL dasotraline, or a pharmaceutically acceptable salt thereof;

b) 0.3 mg/mL to 9.6 mg/mL (5 mM to 50 mM) buffering agent;

c) 1 mg/mL to 2.5 mg/mL preservative;

d) 10 mg/mL to 30 mg/mL cyclodextrin or derivative thereof; and

e) 0.05 mg/mL to 0.2 mg/mL sweetening agent.

In some embodiments, provided is a pharmaceutical composition for oral administration comprising

a) 0.4 mg/mL to 0.6 mg/mL dasotraline, or a pharmaceutically acceptable salt thereof;

b) 0.3 mg/mL to 5.8 mg/mL (5 mM to 30 mM) buffering agent;

c) 1.5 mg/mL to 2.5 mg/mL preservative;

d) 10 mg/mL to 30 mg/mL cyclodextrin or derivative thereof; and

e) 0.05 mg/mL to 0.15 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 0.6 mg/mL to 1.9 mg/mL (10 mM) buffering agent;

c) 2.0 mg/mL±0.25 mg/mL preservative;

d) 25 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 1.5 mg/mL to 4.8 mg/mL (25 mM) buffering agent;

c) 2.0 mg/mL±0.25 mg/mL preservative;

d) 12.5 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 10 mM±2.5 mM buffering agent;

c) 2.0 mg/mL±0.25 mg/mL preservative;

d) 25 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 0.5 mg/mL±0.1 mg/mL dasotraline, or a pharmaceutically acceptable salt thereof;

b) 25 mM±2.5 mM buffering agent;

c) 2.0 mg/mL±0.25 mg/mL preservative;

d) 12.5 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 0.9 mg/mL to 1.1 mg/mL dasotraline, or a pharmaceutically acceptable salt thereof;

b) 0.3 mg/mL to 9.6 mg/mL (5 mM to 50 mM) buffering agent;

c) 1 mg/mL to 2.5 mg/mL preservative;

d) 20 mg/mL to 55 mg/mL cyclodextrin or derivative thereof; and

e) 0.05 mg/mL to 0.2 mg/mL sweetening agent.

In some embodiments, provided is a pharmaceutical composition for oral administration comprising

a) 0.9 mg/mL to 1.1 mg/mL dasotraline, or a pharmaceutically acceptable salt thereof;

b) 0.3 mg/mL to 5.8 mg/mL (5 mM to 30 mM) buffering agent;

c) 1.5 mg/mL to 2.5 mg/mL preservative;

d) 20 mg/mL to 55 mg/mL cyclodextrin or derivative thereof; and

e) 0.05 mg/mL to 0.15 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 0.6 mg/mL to 1.9 mg/mL (10 mM) buffering agent;

c) 2.0 mg/mL±0.25 mg/mL preservative;

d) 50 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 1.5 mg/mL to 4.8 mg/mL (25 mM) buffering agent;

c) 2.0 mg/mL±0.25 mg/mL preservative;

d) 25.0 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 10 mM±2.5 mM buffering agent;

c) 2.0 mg/mL±0.25 mg/mL preservative;

d) 50 mg/mL±5.0 mg/mL cyclodextrin or derivative thereof; and

e) 0.1 mg/mL±0.05 mg/mL sweetening agent.

In some embodiments, provided is a pharmaceutical composition for oral administration comprising

a) 1.0 mg/mL±0.1 mg/mL dasotraline, or a pharmaceutically acceptable salt thereof;

b) 25 mM±2.5 mM buffering agent;

c) 2.0 mg/mL±0.25 mg/mL preservative;

d) 25.0 mg/mL±5.0 mg/mL cyclodextrin or derivative thereof; and

e) 0.1 mg/mL±0.05 mg/mL sweetening agent.

In some embodiments, provided is a pharmaceutical composition comprising

a) 0.4 mg/mL to 1.2 mg/mL dasotraline, or a pharmaceutically acceptable salt thereof;

b) 0.7 mg/mL to 6.7 mg/mL (5 mM to 50 mM) malic acid;

c) 1 mg/mL to 2.5 mg/mL benzoic acid;

d) 10 mg/mL to 60 mg/mL hydroxypropyl-β-cyclodextrin; and

e) 0.05 mg/mL to 0.2 mg/mL sucralose.

In some embodiments, provided is a pharmaceutical composition for oral administration comprising

a) 0.4 mg/mL to 1.2 mg/mL dasotraline, or a pharmaceutically acceptable salt thereof;

b) 0.7 mg/mL to 6.7 mg/mL (5 mM to 50 mM) malic acid;

c) 1 mg/mL to 2.5 mg/mL benzoic acid;

d) 10 mg/mL to 60 mg/mL hydroxypropyl-β-cyclodextrin; and

e) 0.05 mg/mL to 0.2 mg/mL sucralose.

In some embodiments, provided is a pharmaceutical composition for oral administration comprising

a) 0.4 mg/mL to 1.2 mg/mL dasotraline, or a pharmaceutically acceptable salt thereof;

b) 0.7 mg/mL to 6.7 mg/mL (5 mM to 50 mM) malic acid;

c) 1 mg/mL to 2.5 mg/mL benzoic acid;

d) 10 mg/mL to 60 mg/mL hydroxypropyl-β-cyclodextrin; and

e) 0.05 mg/mL to 0.2 mg/mL sucralose in aqueous solution at pH 2.0 to 3.5.

In some embodiments, provided is a pharmaceutical composition for oral administration comprising

a) 0.4 mg/mL to 0.6 mg/mL dasotraline, or a pharmaceutically acceptable salt thereof;

b) 0.7 mg/mL to 6.7 mg/mL (5 mM to 50 mM) malic acid;

c) 1 mg/mL to 2.5 mg/mL benzoic acid;

d) 10 mg/mL to 30 mg/mL hydroxypropyl-β-cyclodextrin; and

e) 0.05 mg/mL to 0.2 mg/mL sucralose.

In some embodiments, provided is a pharmaceutical composition for oral administration comprising

a) 0.4 mg/mL to 0.6 mg/mL dasotraline, or a pharmaceutically acceptable salt thereof;

b) 0.7 mg/mL to 4.0 mg/mL (5 mM to 30 mM) malic acid;

c) 1.5 mg/mL to 2.5 mg/mL benzoic acid;

d) 10 mg/mL to 30 mg/mL hydroxypropyl-β-cyclodextrin; and

e) 0.05 mg/mL to 0.15 mg/mL sucralose.

In some embodiments, provided is a pharmaceutical composition for oral administration comprising

a) 0.5 mg/mL±0.1 mg/mL dasotraline, or a pharmaceutically acceptable salt thereof;

b) 1.34 mg/mL±0.25 mg/mL (10 mM±2.5 mM) malic acid;

c) 2.0 mg/mL±0.25 mg/mL benzoic acid;

d) 25 mg/mL±5.0 mg/mL hydroxypropyl-3-cyclodextrin; and

e) 0.1 mg/mL±0.05 mg/mL sucralose.

In some embodiments, provided is a pharmaceutical composition for oral administration comprising

a) 0.5 mg/mL±0.1 mg/mL dasotraline, or a pharmaceutically acceptable salt thereof;

b) 1.34 mg/mL±0.25 mg/mL (10 mM±2.5 mM) malic acid;

c) 2.0 mg/mL±0.25 mg/mL benzoic acid;

d) 25 mg/mL±5.0 mg/mL hydroxypropyl-β-cyclodextrin; and

e) 0.1 mg/mL±0.05 mg/mL sucralose in aqueous solution at pH 2.6 to 3.2.

In some embodiments, provided is a pharmaceutical composition for oral administration comprising

a) 0.5 mg/mL±0.1 mg/mL dasotraline, or a pharmaceutically acceptable salt thereof;

b) 3.35 mg/mL±0.25 mg/mL (25 mM±2.5 mM) malic acid;

c) 2.0 mg/mL±0.25 mg/mL benzoic acid;

d) 12.5 mg/mL±5.0 mg/mL hydroxypropyl-β-cyclodextrin; and

e) 0.1 mg/mL±0.05 mg/mL sucralose.

In some embodiments, provided is a pharmaceutical composition for oral administration comprising

a) 0.5 mg/mL±0.1 mg/mL dasotraline, or a pharmaceutically acceptable salt thereof;

b) 3.35 mg/mL±0.25 mg/mL (25 mM±2.5 mM) malic acid;

c) 2.0 mg/mL±0.25 mg/mL benzoic acid;

d) 12.5 mg/mL±5.0 mg/mL hydroxypropyl-β-cyclodextrin; and

e) 0.1 mg/mL±0.05 mg/mL sucralose in aqueous solution at pH 2.2 to 2.8.

In some embodiments, provided is a pharmaceutical composition for oral administration comprising

a) 0.9 mg/mL to 1.1 mg/mL dasotraline, or a pharmaceutically acceptable salt thereof;

b) 0.7 mg/mL to 6.7 mg/mL (5 mM to 50 mM) malic acid;

c) 1 mg/mL to 2.5 mg/mL benzoic acid;

d) 20 mg/mL to 55 mg/mL hydroxypropyl-β-cyclodextrin; and

e) 0.05 mg/mL to 0.2 mg/mL sucralose.

In some embodiments, provided is a pharmaceutical composition for oral administration comprising

a) 0.9 mg/mL to 1.1 mg/mL dasotraline, or a pharmaceutically acceptable salt thereof;

b) 0.7 mg/mL to 4.0 mg/mL (5 mM to 30 mM) malic acid;

c) 1.5 mg/mL to 2.5 mg/mL benzoic acid;

d) 20 mg/mL to 55 mg/mL hydroxypropyl-β-cyclodextrin; and

e) 0.05 mg/mL to 0.15 mg/mL sucralose.

In some embodiments, provided is a pharmaceutical composition for oral administration comprising

a) 1.0 mg/mL±0.1 mg/mL dasotraline, or a pharmaceutically acceptable salt thereof;

b) 1.34 mg/mL±0.25 mg/mL (10 mM±2.5 mM) malic acid;

c) 2.0 mg/mL±0.25 mg/mL benzoic acid;

d) 50 mg/mL±5.0 mg/mL hydroxypropyl-3-cyclodextrin; and

e) 0.1 mg/mL±0.05 mg/mL sucralose.

In some embodiments, provided is a pharmaceutical composition for oral administration comprising

a) 1.0 mg/mL±0.1 mg/mL dasotraline, or a pharmaceutically acceptable salt thereof;

b) 1.34 mg/mL±0.25 mg/mL (10 mM±2.5 mM) malic acid;

c) 2.0 mg/mL±0.25 mg/mL benzoic acid;

d) 50 mg/mL±5.0 mg/mL hydroxypropyl-3-cyclodextrin; and

e) 0.1 mg/mL±0.05 mg/mL sucralose in aqueous solution at pH 2.6 to 3.2.

In some embodiments, provided is a pharmaceutical composition for oral administration comprising

a) 1.0 mg/mL±0.1 mg/mL dasotraline, or a pharmaceutically acceptable salt thereof;

b) 3.35 mg/mL±0.25 mg/mL (25 mM±2.5 mM) malic acid;

c) 2.0 mg/mL±0.25 mg/mL benzoic acid;

d) 25.0 mg/mL±5.0 mg/mL hydroxypropyl-β-cyclodextrin; and

e) 0.1 mg/mL±0.05 mg/mL sucralose.

In some embodiments, provided is a pharmaceutical composition for oral administration comprising

a) 1.0 mg/mL±0.1 mg/mL dasotraline, or a pharmaceutically acceptable salt thereof;

b) 3.35 mg/mL±0.25 mg/mL (25 mM±2.5 mM) malic acid;

c) 2.0 mg/mL±0.25 mg/mL benzoic acid;

d) 25.0 mg/mL±5.0 mg/mL hydroxypropyl-β-cyclodextrin; and

e) 0.1 mg/mL±0.05 mg/mL sucralose in aqueous solution at pH 2.2 to 2.8.

In some embodiments, provided is a pharmaceutical composition for oral administration comprising

a) 1.0 mg/mL±0.1 mg/mL dasotraline, or a pharmaceutically acceptable salt thereof;

b) 3.35 mg/mL±0.25 mg/mL (25 mM±2.5 mM) malic acid;

c) 2.0 mg/mL±0.25 mg/mL benzoic acid;

d) 25.0 mg/mL±5.0 mg/mL hydroxypropyl-β-cyclodextrin;

e) 0.1 mg/mL±0.05 mg/mL sucralose;

f) optionally coloring agent; and

g) sufficient alkali or alkaline earth metal salt to provide a pH between 2.2 and 2.8.

As described in the Examples provided herein, the ratio of cyclodextrin, or derivative thereof, to preservative may be relevant to a pharmaceutical formulation's function. For example, the ratio of cyclodextrin, or derivative thereof, to preservative may result in undesirable complexation or increase the risk of microbial growth during storage/distribution. In some embodiments, the ratio of cyclodextrin or derivative thereof to preservative is below 25:1. In some embodiments, the ratio of hydroxypropyl-β-cyclodextrin to benzoic acid is below 25:1.

A pharmaceutical composition disclosed herein may further comprise purified water.

In some embodiments, provided are pharmaceutical compositions comprising dasotraline or a pharmaceutically acceptable salt thereof, having a decreased bitterness or paresthesic sensation, wherein the pharmaceutical composition further comprises a buffering agent and a cyclodextrin or derivative thereof. In some embodiments, provided are pharmaceutical compositions comprising dasotraline or a pharmaceutically acceptable salt thereof, having a decreased bitterness or paresthesic sensation, wherein the pharmaceutical composition further comprises a buffering agent and a cyclodextrin or derivative thereof, and optionally a preservative. In some embodiments, provided are pharmaceutical compositions comprising dasotraline or a pharmaceutically acceptable salt thereof, having a decreased bitterness or paresthesic sensation, wherein the pharmaceutical composition further comprises a buffering agent and a cyclodextrin or derivative thereof, and optionally a sweetening agent. In some embodiments, provided are pharmaceutical compositions comprising dasotraline or a pharmaceutically acceptable salt thereof, having a decreased bitterness or paresthesic sensation, wherein the pharmaceutical composition further comprises a buffering agent and a cyclodextrin or derivative thereof, and optionally a preservative and a sweetening agent.

In some embodiments, provided are pharmaceutical compositions comprising dasotraline or a pharmaceutically acceptable salt thereof, having a decreased bitterness or paresthesic sensation, wherein the pharmaceutical composition further comprises a buffering agent, a preservative, a cyclodextrin or derivative thereof, and a sweetening agent as disclosed herein.

In some embodiments, provided are pharmaceutical compositions prepared by processes disclosed herein. In some embodiments, provided is a pharmaceutical composition comprising dasotraline, or a pharmaceutically acceptable salt thereof, wherein the pharmaceutical composition is prepared by first dissolving a buffering agent, a preservative, a cyclodextrin or derivative thereof, in water and second adding dasotraline, or a pharmaceutically acceptable salt thereof. In some embodiments, provided is a pharmaceutical composition comprising dasotraline, or a pharmaceutically acceptable salt thereof, wherein the pharmaceutical composition is solution prepared by first dissolving a buffering agent, a preservative, a cyclodextrin or derivative thereof, in water, second adding dasotraline, or a pharmaceutically acceptable salt thereof, and third passing the solution through a filter. In some embodiments, provided are processes of preparing a therapeutically effective oral solution comprising (a) dissolving one or more pharmaceutically acceptable excipients in purified water, (b) adding and dissolving dasotraline, or a pharmaceutically acceptable salt thereof, and (c) passing the solution through a filter. In some embodiments, the filter is 0.1 μm, 0.2 μm, 0.3 μm, 0.4 μm, 0.5 μm, 0.6 μm, 0.7 μm, 0.8 μm, 0.9 μm, 1.0 μm, 1.5 μm, 2.0 μm, 2.5 μm, 3.0 μm, 3.5 μm, 4.0 μm, 4.5 μm, 5.0 μm, 5.5 μm, or 6.0 μm. In some embodiments, the filter is 0.45 μm, 1.0 μm, or 5.0 μm.

Methods

Dasotraline has properties helpful in treating Feeding and Eating Disorders, such as binge eating disorder as shown in clinical trial nos. NCT02795637 and NCT02564588, and Neurodevelopmental Disorders, such as attention-deficit hyperactivity disorder, as shown in clinical trial nos. NCT02795637, NCT02195167, NCT02734693, NCT02276209, NCT02428088, and NCT02457819.

Provided are methods of treating Feeding and Eating Disorders comprising administering a pharmaceutical composition disclosed herein. In some embodiments, the Feeding and Eating Disorder is pica, rumination disorder, avoidant/restrictive food intake disorder, anorexia nervosa, bulimia nervosa, binge eating disorder, other specified feeding or eating disorder, or unspecified feeding or eating disorder. In some embodiments, the Feeding and Eating Disorder is binge eating disorder.

Provided are methods of treating Neurodevelopmental Disorders comprising administering a pharmaceutical composition disclosed herein. In some embodiments, the Neurodevelopmental Disorder is Intellectual Disability Disorder, Global Developmental Delay, Unspecified Intellectual Disability, Communication Disorder, Language Disorder, Speech Sound Disorder, Childhood-Onset Fluency Disorder (Stuttering), Social (Pragmatic) Communication Disorder, Unspecified Communication Disorder, Autism Spectrum Disorder, Attention-Deficit/Hyperactivity Disorder (ADHD), Other Specified Attention/Hyperactivity Disorder, Unspecified Attention-Deficit/Hyperactivity Disorder, Specific Learning Disorder, Motor Disorder, Developmental Coordination Disorder, Stereotypic Movement Disorder, Tic Disorder, Other Specified Tic Disorder, Unspecified Tic Disorder, Other Neurodevelopmental Disorders, Other Specified Neurodevelopmental Disorder, or Unspecified Neurodevelopmental Disorder. In some embodiments, the Neurodevelopmental Disorder is Attention-Deficit/Hyperactivity Disorder (ADHD).

Provided are methods of treating disorders disclosed herein comprising administering a pharmaceutical composition disclosed herein, wherein the method comprises using a cup, a dosing spoon, a dropper, or an oral syringe. In some embodiments, provided are methods of treating a disorder disclosed herein comprising filling a cup with a pharmaceutical composition disclosed herein and delivering the pharmaceutical composition to a subject in need thereof. In some embodiments, provided are methods of treating a disorder disclosed herein comprising filling a dosing spoon with a pharmaceutical composition disclosed herein and delivering the pharmaceutical composition to a subject in need thereof. In some embodiments, provided are methods of treating a disorder disclosed herein comprising filling a dropper with a pharmaceutical composition disclosed herein and delivering the pharmaceutical composition to a subject in need thereof. In some embodiments, provided are methods of treating a disorder disclosed herein comprising filling an oral syringe with a pharmaceutical composition disclosed herein and delivering the pharmaceutical composition to a subject in need thereof. In some embodiments, the method comprising storing the pharmaceutical composition in a container that limits exposure to light. In some embodiments, the container allows exposure to less than 0.6 million lux·hr of light. In some embodiments, the container allows exposure to less than 1.2 million lux·hr of light. In some embodiments, the container allows exposure to less than 0.6 million lux·hr of light over a period of at least four weeks. In some embodiments, the container allows exposure to less than 1.2 million lux·hr of light over a period of at least four weeks.

Provided are methods of decreasing the bitterness or paresthesic sensation of a pharmaceutical composition comprising dasotraline, or a pharmaceutically acceptable salt thereof, wherein the method comprises preparing the pharmaceutical composition with a cyclodextrin or a derivative thereof as disclosed herein. In some embodiments, provided are methods of decreasing the bitterness or paresthesic sensation of a pharmaceutical composition comprising dasotraline, or a pharmaceutically acceptable salt thereof, wherein the method comprises preparing the pharmaceutical composition with a cyclodextrin or a derivative thereof and a sweetening agent as disclosed herein. In some embodiments, provided are methods of decreasing the bitterness or paresthesic sensation of a pharmaceutical composition comprising dasotraline, or a pharmaceutically acceptable salt thereof, wherein the method comprises preparing the pharmaceutical composition with a cyclodextrin or a derivative thereof and a sweetening agent as disclosed herein and administering to a subject in need thereof.

In some embodiments, the decrease in bitterness or paresthesic sensation is measured by a Flavor Profile Method, an e-tongue test, or an in vivo sensory evaluation. In some embodiments, the bitterness or paresthesic sensation of dasotraline is compared to the bitterness or paresthesic sensation of a pharmaceutical composition disclosed herein. In some embodiments, the bitterness or paresthesic sensation of dasotraline HCl is compared to the bitterness or paresthesic sensation of a pharmaceutical composition disclosed herein.

In some embodiments, the decrease in bitterness or paresthesic sensation is measured by a Flavor Profile Method. In some embodiments, the bitterness or paresthesic sensation is measured by a Flavor Profile Method as described by Keane, P. The Flavor Profile Method. In C. Hootman (Ed.), Manual on Descriptive Analysis Testing for Sensory Evaluation ASTM Manual Series: MNL 13. Baltimore, Md. (1992). In some embodiments, the bitterness or paresthesic sensation of dasotraline is compared to the bitterness or paresthesic sensation of a pharmaceutical composition disclosed herein, wherein the bitterness or paresthesic sensation is measured by a Flavor Profile Method using Mouthfeel, Off-Notes, and Aftertaste. In some embodiments, the bitterness or paresthesic sensation of dasotraline HCl is compared to the bitterness or paresthesic sensation of a pharmaceutical composition disclosed herein, wherein the bitterness or paresthesic sensation is measured by a Flavor Profile Method using Mouthfeel, Off-Notes, and Aftertaste. In some embodiments, the bitterness or paresthesic sensation of dasotraline is compared to the bitterness or paresthesic sensation of a pharmaceutical composition disclosed herein, wherein the bitterness or paresthesic sensation is measured by a Flavor Profile Method using Mouthfeel, Off-Notes, and Aftertaste values and the pharmaceutical composition disclosed herein decreases the Mouthfeel, Off-Notes, or Aftertaste values by ¼, ½, ¾ or 1 compared to the Mouthfeel, Off-Notes, or Aftertaste values of dasotraline. In some embodiments, the bitterness or paresthesic sensation of dasotraline HCl is compared to the bitterness or paresthesic sensation of a pharmaceutical composition disclosed herein, wherein the bitterness is measured by a Flavor Profile Method using Mouthfeel, Off-Notes, and Aftertaste values and the pharmaceutical composition disclosed herein decreases the Mouthfeel, Off-Notes, or Aftertaste values by ¼, ½, ¾ or 1 compared to the Mouthfeel, Off-Notes, or Aftertaste values of dasotraline HCl.

In some embodiments, the mouthfeel value evaluates any one or more of glycyrrhiza (licorice) mouthfeel, tongue sting mouthfeel, tongue burn mouthfeel, cooling mouthfeel, drying mouthfeel, or chalky mouthfeel; or combination thereof. In some embodiments, the off-note value evaluates any one or more of dairy sour aromatic, bitter, or metallic aromatic; or combination thereof. In some embodiments, the aftertaste value evaluates sweet or sour; or combination thereof.

In some embodiments, the Flavor Profile Method is performed over 30 minutes. In some embodiments, the Flavor Profile Method values (e.g. Mouthfeel, Off-Notes, Aftertaste) are evaluated immediately, and every 1 minute following for a total of 30 minutes.

In some embodiments, the decrease in bitterness is measured by an e-tongue (electronic tongue) test. Non-limiting examples of e-tongue systems include the Astree II e-tongue, Insent SA 402B, Insent TS-5000Z, and Higuchi E-Tongue. In some embodiments, the bitterness is measured by an e-tongue test with a sensor selected from the group consisting of BT0, AN0, C00, and AE1. In some embodiments, the bitterness is measured by an e-tongue test using a BT0 sensor and a AN0 sensor. In some embodiments, the bitterness is measured by the output value as measured in volts by an e-tongue test. In some embodiments, the bitterness is measured by the output value as measured in mV by an e-tongue test. In some embodiments, the bitterness of a pharmaceutical composition disclosed herein is decreased by at least about 5%, 10%, 15%, 20%, 25%, 30%, 35%, 40%, 45%, 50%, 55%, 60%, 65%, 70%, or 75% compared to the bitterness of dasotraline. In some embodiments, the bitterness of a pharmaceutical composition disclosed herein is decreased by at least about 5%, 10%, 15%, 20%, 25%, 30%, 35%, 40%, 45%, 50%, 55%, 60%, 65%, 70%, or 75% compared to the bitterness of dasotraline HCl.

In some embodiments, the decrease in bitterness or paresthesic sensation is measured by an in vivo sensory evaluation study.

Provided are pharmaceutical composition as disclosed herein for use in treating Feeding and Eating Disorders. In some embodiments, the Feeding and Eating Disorder is pica, rumination disorder, avoidant/restrictive food intake disorder, anorexia nervosa, bulimia nervosa, binge eating disorder, other specified feeding or eating disorder, or unspecified feeding or eating disorder. In some embodiments, the Feeding and Eating Disorder is binge eating disorder.

Provided are pharmaceutical composition as disclosed herein for use in treating Neurodevelopmental Disorders comprising administering a pharmaceutical composition disclosed herein. In some embodiments, the Neurodevelopmental Disorder is Intellectual Disability Disorder, Global Developmental Delay, Unspecified Intellectual Disability, Communication Disorder, Language Disorder, Speech Sound Disorder, Childhood-Onset Fluency Disorder (Stuttering), Social (Pragmatic) Communication Disorder, Unspecified Communication Disorder, Autism Spectrum Disorder, Attention-Deficit/Hyperactivity Disorder (ADHD), Other Specified Attention/Hyperactivity Disorder, Unspecified Attention-Deficit/Hyperactivity Disorder, Specific Learning Disorder, Motor Disorder, Developmental Coordination Disorder, Stereotypic Movement Disorder, Tic Disorder, Other Specified Tic Disorder, Unspecified Tic Disorder, Other Neurodevelopmental Disorders, Other Specified Neurodevelopmental Disorder, or Unspecified Neurodevelopmental Disorder. In some embodiments, the Neurodevelopmental Disorder is Attention-Deficit/Hyperactivity Disorder (ADHD).

Provided are pharmaceutical composition as disclosed herein for use as a medicament for treating Feeding and Eating Disorders. In some embodiments, the Feeding and Eating Disorder is pica, rumination disorder, avoidant/restrictive food intake disorder, anorexia nervosa, bulimia nervosa, binge eating disorder, other specified feeding or eating disorder, or unspecified feeding or eating disorder. In some embodiments, the Feeding and Eating Disorder is binge eating disorder.

Provided are pharmaceutical composition as disclosed herein for use as a medicament for treating Neurodevelopmental Disorders comprising administering a pharmaceutical composition disclosed herein. In some embodiments, the Neurodevelopmental Disorder is Intellectual Disability Disorder, Global Developmental Delay, Unspecified Intellectual Disability, Communication Disorder, Language Disorder, Speech Sound Disorder, Childhood-Onset Fluency Disorder (Stuttering), Social (Pragmatic) Communication Disorder, Unspecified Communication Disorder, Autism Spectrum Disorder, Attention-Deficit/Hyperactivity Disorder (ADHD), Other Specified Attention/Hyperactivity Disorder, Unspecified Attention-Deficit/Hyperactivity Disorder, Specific Learning Disorder, Motor Disorder, Developmental Coordination Disorder, Stereotypic Movement Disorder, Tic Disorder, Other Specified Tic Disorder, Unspecified Tic Disorder, Other Neurodevelopmental Disorders, Other Specified Neurodevelopmental Disorder, or Unspecified Neurodevelopmental Disorder. In some embodiments, the Neurodevelopmental Disorder is Attention-Deficit/Hyperactivity Disorder (ADHD).

Provided are pharmaceutical composition as disclosed herein for use in the manufacture of a medicament for treating Feeding and Eating Disorders. In some embodiments, the Feeding and Eating Disorder is pica, rumination disorder, avoidant/restrictive food intake disorder, anorexia nervosa, bulimia nervosa, binge eating disorder, other specified feeding or eating disorder, or unspecified feeding or eating disorder. In some embodiments, the Feeding and Eating Disorder is binge eating disorder.

Provided are pharmaceutical composition as disclosed herein for use in the manufacture of a medicament for treating Neurodevelopmental Disorders comprising administering a pharmaceutical composition disclosed herein. In some embodiments, the Neurodevelopmental Disorder is Intellectual Disability Disorder, Global Developmental Delay, Unspecified Intellectual Disability, Communication Disorder, Language Disorder, Speech Sound Disorder, Childhood-Onset Fluency Disorder (Stuttering), Social (Pragmatic) Communication Disorder, Unspecified Communication Disorder, Autism Spectrum Disorder, Attention-Deficit/Hyperactivity Disorder (ADHD), Other Specified Attention/Hyperactivity Disorder, Unspecified Attention-Deficit/Hyperactivity Disorder, Specific Learning Disorder, Motor Disorder, Developmental Coordination Disorder, Stereotypic Movement Disorder, Tic Disorder, Other Specified Tic Disorder, Unspecified Tic Disorder, Other Neurodevelopmental Disorders, Other Specified Neurodevelopmental Disorder, or Unspecified Neurodevelopmental Disorder. In some embodiments, the Neurodevelopmental Disorder is Attention-Deficit/Hyperactivity Disorder (ADHD).

Provided are uses of a pharmaceutical composition as disclosed herein for the manufacture of a medicament for the treatment of Feeding and Eating Disorders. In some embodiments, the Feeding and Eating Disorder is pica, rumination disorder, avoidant/restrictive food intake disorder, anorexia nervosa, bulimia nervosa, binge eating disorder, other specified feeding or eating disorder, or unspecified feeding or eating disorder. In some embodiments, the Feeding and Eating Disorder is binge eating disorder.

Provided are uses of a pharmaceutical composition as disclosed herein for the manufacture of a medicament for the treatment of Neurodevelopmental Disorders comprising administering a pharmaceutical composition disclosed herein. In some embodiments, the Neurodevelopmental Disorder is Intellectual Disability Disorder, Global Developmental Delay, Unspecified Intellectual Disability, Communication Disorder, Language Disorder, Speech Sound Disorder, Childhood-Onset Fluency Disorder (Stuttering), Social (Pragmatic) Communication Disorder, Unspecified Communication Disorder, Autism Spectrum Disorder, Attention-Deficit/Hyperactivity Disorder (ADHD), Other Specified Attention/Hyperactivity Disorder, Unspecified Attention-Deficit/Hyperactivity Disorder, Specific Learning Disorder, Motor Disorder, Developmental Coordination Disorder, Stereotypic Movement Disorder, Tic Disorder, Other Specified Tic Disorder, Unspecified Tic Disorder, Other Neurodevelopmental Disorders, Other Specified Neurodevelopmental Disorder, or Unspecified Neurodevelopmental Disorder. In some embodiments, the Neurodevelopmental Disorder is Attention-Deficit/Hyperactivity Disorder (ADHD).

### EXAMPLES

Development of a pharmaceutical composition provides for a plurality of considerations, such as route of administration (e.g. enteral, parenteral, topical, etc.), dosage form (solid—tablet, capsule, etc.; liquid—solution, suspension, syrup, etc.), strength of therapeutic component(s) (1-1,000 mg), non-therapeutic component(s) and their respective amounts, and each of these considerations require additional considerations such as stability, degradation, sensitivity to light, solubility, taste if administered enterally, palatability, pH, skin irritability, microbial growth, etc. Discovering and developing a pharmaceutical composition that addresses these considerations must also maintain the desired therapeutic effect. Thus, in addressing an unmet need of treating populations with difficulty ingesting solid form pharmaceutical compositions (e.g. pediatrics, elderly subjects, etc.), a liquid form pharmaceutical composition for oral administration that provides a therapeutic effect while employing such considerations is desired. In the Examples hereinafter described, the unit of % used for preservatives and taste-masking agents (cyclodextrins and/or sweetening agents) means weight percentage (percentage by weight) and, for example, the 0.01%, 0.1%, 0.15%, 0.2%, 0.5%, 1%, 1.25%, 1.5%, 2.5% 5% and 10% could naturally be converted to 0.1 mg/mL, 1 mg/mL, 1.5 mg/mL, 2 mg/mL, 5 mg/mL, 10 mg/mL, 12.5 mg/mL, 15 mg/mL, 25 mg/mL, 50 mg/mL, 100 mg/mL.

### Example 1: Taste of Dasotraline (Adult Panel)

As described herein, dasotraline provides a complication in developing an orally administered pharmaceutical composition due to the naturally unpleasant taste and/or paresthesic sensation provided by dasotraline. This was discovered, in part, during a human taste study using the Flavor Profile Method of descriptive sensory analysis (Keane, P. The Flavor Profile Method. In C. Hootman (Ed.), Manual on Descriptive Analysis Testing for Sensory Evaluation ASTM Manual Series: MNL 13. Baltimore, Md. (1992)). The study involved seven experienced pharmaceutical sensory panelists. Each panelist was provided a capsule containing dasotraline in the following order and amount: (1) 2 mg (2) 2 mg (3) 4 mg (4) 4 mg (5) 6 mg (6) 6 mg (7) 8 mg (8) 8 mg. The panelists opened a single capsule shell and emptied the contents onto a piece of weighing paper. Starting at the same time, the panelists poured the sample directly into their mouths, swished the contents around the oral cavity for 10 seconds and expectorated. During this time the panelists independently evaluated and recorded the initial flavor characteristics and the aftertaste characteristics at periodic intervals out to 30 minutes. This process was repeated for each strength.

The flavor profile for the 2 mg capsule included a bitter basic taste that lingered at patient-perceptible levels for the first five minutes of aftertaste and a tongue sting and metallic aromatics that lingered for 3 minutes. The flavor profile for the 4 mg capsule was similar to the 2 mg capsule, but was more intense and lingered longer at patient-perceptible levels. The tongue sting mouthfeel effect was more “burning” in character and a lingering glycyrrhiza (cloying, licorice-like sensation) was perceived. The flavor profiles for the 6 mg capsule and the 8 mg capsule were similar to the 4 mg capsule, but persisted at patient-perceptible intensities for longer periods of time.

Since all capsule strengths produced patient-perceptible undesirable tastes and/or sensations, the natural taste profile of dasotraline could discourage patients from ingesting the quantity needed for therapeutic efficacy.

### Example 2: Solubility

In an attempt to mask the naturally bitter taste of dasotraline, a solution was explored. To make a solution, one must attain sufficient solubility to achieve efficacious levels of an active pharmaceutical ingredient (API) while avoiding precipitation. In the case of dasotraline HCl, the solubility in water is a function of pH, with maximal solubility in the window between pH 2.0 and pH 3.0. As shown in Table 1, dasotraline HCl provided a minimum solubility at 0.4 mg/mL at pH 5.0 in 50 mM citrate buffer and a maximum solubility at 2.2 mg/mL at pH 2.7 in 50 mM citrate buffer.

TABLE 1
Solubility of dasotraline HCl in citrate buffer

| | | Measured |
|---|---|---|
| Solvent pH | Final pH | solubility(mg/mL*) |
| 2.2 | 2.2 | 1.9 |
| 2.7 | 2.7 | 2.2 |
| 3.0 | 3.0 | 2.1 |
| 3.5 | 3.4 | 1.3 |
| 4.0 | 3.9 | 0.8 |
| 4.5 | 4.4 | 0.6 |
| 5.0 | 4.9 | 0.4 |

*as free base

### Example 3: Stability (Buffering Agent)

A solution must be stable for commercially reasonable periods. To achieve a pH of 2-3 for a solution comprising a compound that is itself a weak base, such as dasotraline HCl, a buffer may be used. In experimenting with buffering agents such as tartrate and citrate, precipitation of dasotraline HCl was discovered. Specifically, precipitation of dasotraline HCl at pH 3 was discovered with tartrate buffer and, similarly, citrate buffer evinced precipitation of dasotraline HCl immediately at pH 5 and after four weeks at pH 3.5 at 60° C. It was surprisingly found that a malate buffer provided clear solutions after four weeks at up to pH 4 at 60° C. The malate buffer at 10 mM and 25 mM (pH 2.5-pH 4.0) provided clear solutions under all test conditions, including four weeks at 60° C. However, the higher buffer amount showed impurities >0.1% after four weeks at pH>3 at 60° C., whereas the 10 mM buffer showed no detectable impurities after four weeks at pH≤3.5 at 60° C. An acetate buffered solution showed similar stability, but the acetate buffer added a strong sour taste and flavor, which exacerbates the natural taste problem of dasotraline HCl.

### Example 4: Stability (Preservative)

In addition to chemical/physical stability, the formulation must remain free of microbial growth for commercially reasonable periods. Several common preservatives failed when combined with dasotraline HCl and malate buffer. For example, sodium benzoate induced precipitation at 0.2%. 5% propylene glycol was effective up to four weeks at 60° C., but gave rise to >0.1% impurities on further stress. 0.2% Benzoic acid provided a stable solution under all conditions tested. Formulations comprising 0.15-0.2% benzoic acid showed (bacteria—Escherichia coli, Pseudomonas aeruginosa, and Staphylococcus aurerus) not less than a 1.0 log reduction from the initial count at 14 days, and no increase from the 14 day count at 28 days and (yeast and molds—Candida albicans and Aspergillus niger) no increase from the initial calculated count at 14 and 28 days.

### Example 5: Stability (Taste-Masking Agent)

As described in Example 1, a pharmaceutical composition comprising dasotraline provides an additional complication of an unpleasant taste and/or paresthesic sensation. In an attempt to overcome this complication, while also considering stability and other factors, solutions comprising cyclodextrin or a derivative thereof, or sucralose, or xanthan gum or carrageenam were explored. It was found that the amount of unspecified impurities increased in a manner that was dependent on HPBC concentration. Moreover, solutions comprising Xanthan gum or carrageenan were found to cause dasotraline to precipitate out of solution. Impurities in samples containing 2.5-10% HPBC or 0.1% sucralose and 10 mM malic acid were satisfactory (below reporting limit).

### Example 6: Taste of Formulations Comprising Dasotraline and Cyclodextrin or Derivatives Thereof (e-Tongue Test)

The taste-masking effect of HPBC was evaluated by an e-tongue test. Test solutions were prepared by mixing components, filtering, diluting with 10 mM or 25 mM malic acid to adjust dasotraline concentration to 0.2 mg/mL (as free base). Sensor screening was conducted and BT0 (Saltiness and Bitterness) and AN0 (Saltiness, Sourness, and Bitterness) sensors were selected to evaluate the bitterness of the dasotraline-containing formulations as shown in Table 2.

TABLE 2
Formulations Comprising Dasotraline and
Cyclodextrin or Derivatives thereof

| Formu- | | | Sweetening | Buffering | |
|---|---|---|---|---|---|
| lation | Cyclodextrin | | Agent | Agent | Dasotraline |
| F1 | 0% | HPBC | — | 10 mM Malic | 1.0 mg/mL |
| F2 | 2.5% | HPBC | 0.01% | Acid | |
| F3 | 5% | HPBC | Sucralose | | |
| F4 | 0% | βCD | — | 25 mM Malic | |
| F5 | 1% | βCD | 0.01% | Acid | |
| F6 | 1.5% | βCD | Sucralose | | |

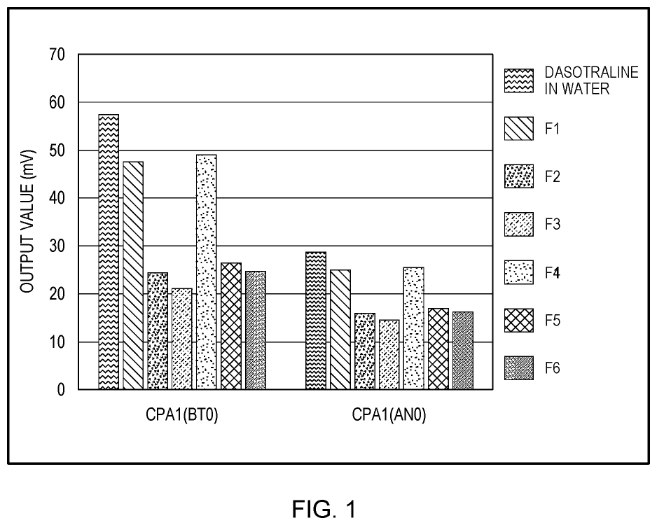

Results of the e-tongue test are shown in FIG. 1, where formulations containing HPBC (F2 and F3) and βCD (F5 and F6) demonstrated lower bitterness compared to that of the formulations without HPBC (F1 and F4). HPBC and βCD were effective in reducing or eliminating the perceived bitterness of dasotraline.

### Example 7: Microbial Growth

A further complexity in designing an aqueous solutions is overall chemical stability and freedom from microbial growth. Preservative efficacy of prototype formulations was evaluated, according to United States Pharmacopeia (USP) 40 “<51> Antimicrobial Effectiveness Testing”. Test formulations were prepared by mixing components shown in Table 3. All formulations passed the criteria of antimicrobial effectiveness required for category 3 products (see Table 3 below). However, undesirable mold growth was found in the samples containing 0.05% and 0.1% benzoic acid with 5% HPBC, and viable counts of Aspergillus brasiliensis were found in the formulation containing 5% HPBC and 0.15% benzoic acid after 28 days from inoculation. Antimicrobial efficacy was improved by altering the ratio of benzoic acid to HPBC by increasing the concentration of benzoic acid and/or reducing the concentration of HPBC.

TABLE 3
Criteria for tested microorganisms

| | | Criteria |
|---|---|---|
| Organisms | | (Product category: Category 3) |
| Bacteria | Escherichia coli | Not less than 1.0 log reduction |
| | Pseudomonas aeruginosa | from the initial count at 14 |
| | Staphylococcus aureus | days, and no increase from the 14 |
| | | days' count at 28 days |
| Yeast and | Candida albicans | No increase from the initial |
| Mold | Aspergillus niger | calculated count at 14 and 28 days |

From the test results, the suitable concentration of benzoic acid to be added in the formulation was considered to be 0.2% for a formulation containing 5% HPBC and 0.15-0.2% for the formulation containing 2.5% HPBC. To minimize the risk of contamination and microorganisms' growth during storage and clinical use, 0.2% was selected as benzoic acid concentration. All prototype formulations passed the criteria of antimicrobial effectiveness required for category 3 products and showed high log reduction against to the test microorganisms.

Based on the foregoing studies, formulations containing 1.0 mg/mL and 0.5 mg/mL of dasotraline are shown in Table 4.

TABLE 4
Pharmaceutical Compositions Comprising Dasotraline

| | | Sweetening | Buffering | | |
|---|---|---|---|---|---|
| | Cyclodextrin | Agent | agent | Preservative | Dasotraline |
| F10 | 5% HPBC | 0.01% | 10 mM Malic | 0.20% | 1.0 mg/mL |
| | | Sucralose | acid | Benzoic acid | |
| F11 | 2.5% HPBC | | 25 mM Malic | | 1.0 mg/mL |
| | | | acid | | |
| F12 | 2.5% HPBC | | 10 mM Malic | | 0.5 mg/mL |
| | | | acid | | |
| F13 | 1.25% HPBC | | 25 mM Malic | | 0.5 mg/mL |
| | | | acid | | |

The formulations described in Table 4 were prepared by dissolving HPBC, DL-malic acid, sucralose, and benzoic acid in purified water with mixing. The solution was filtered through a 0.2 μm filter and sealed in glass bottles. Under some circumstances it may be found desirable to add small amounts (less than 0.07%) of coloring and/or additional taste masking agents.

### Example 8: Taste Masking (In Vivo Sensory Studies)

Formulations of Table 4 (Example 7) were evaluated for taste and/or paresthesic sensation using the method described in Example 1. For the evaluations of the formulations in Table 4, ten experienced panelists participated in the study. Panelists evaluated the samples using the Flavor Profile Method (Keane, op. cit.). Flavor Profile is used to identify, characterize and quantify sensory attributes such as taste, aroma, texture and mouthfeel. Prior to tasting, the panelists wore disposable lab coats, gloves and goggles to avoid exposure to the drug active, which is known to be an ocular irritant. Each panelist was given a 5 mL sample of each solution in a one-ounce plastic cup. Starting at the same time, the panelists poured the sample directly in to their mouths, swished the contents around the oral cavity for 10 seconds and expectorated. During this time the panelists independently evaluated and recorded the initial flavor characteristics. The panelists then independently evaluated and recorded the aftertaste characteristics at periodic intervals out to 30 minutes as flavor persisted. The panelists recited their individual results and a preliminary Flavor Profile was generated for the sample. The steps were repeated for a second sample using the preliminary Flavor Profile as a guide, with the panelists making any necessary modifications. The panelists recited their individual results and a final Flavor Profile was developed for the sample.

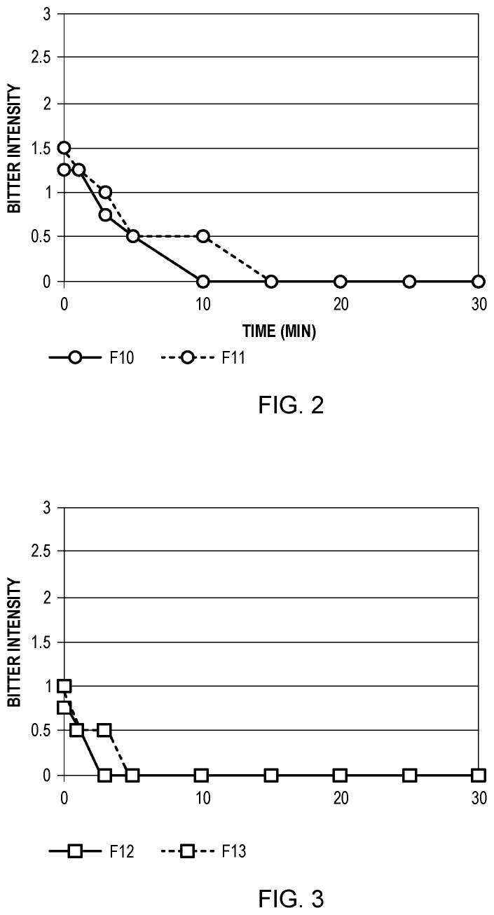

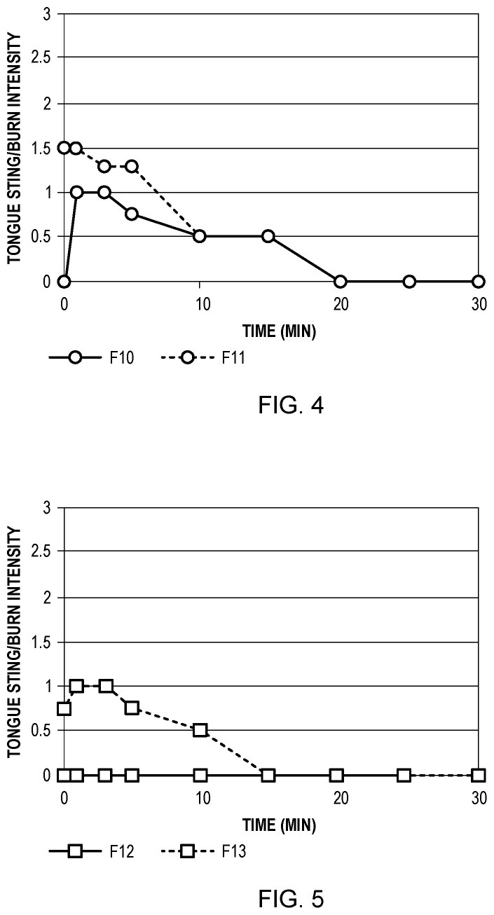

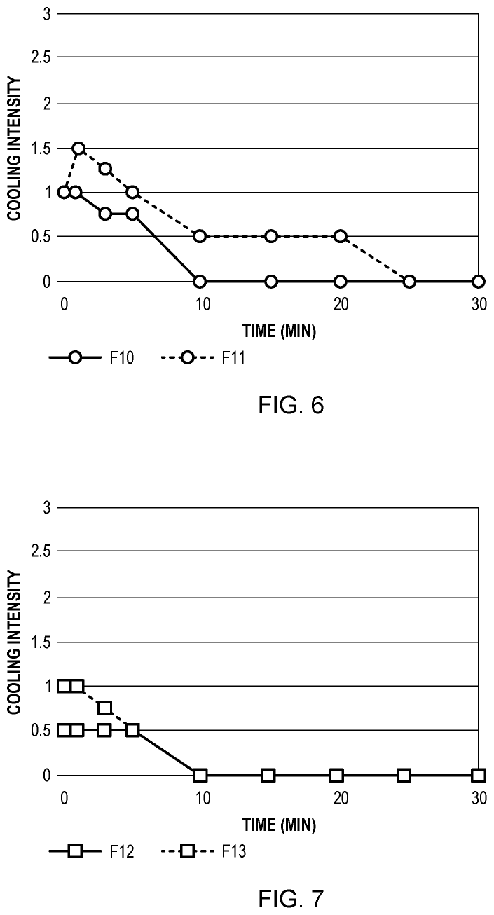

From the study described in Example 1, bitter basic taste and unpleasant mouthfeel (tongue sting/burn, cooling, glycyrrhiza) were the attributes that required amelioration. On a scale of 1-5, aversive sensory characteristics greater than 1 are clearly perceptible to patients and are often found to be unacceptable. As can be seen in FIG. 3, at the 0.5 mg/mL dasotraline strength (formulations 12 and 13 in Table 4), the bitterness is below the level of patient perception (<1) and, as can be seen in FIG. 2, slightly above this level for the 1 mg/mL strength. At both dasotraline strengths, the differences in amount of cyclodextrin had a small, but positive effect on the bitterness profile. For the tongue sting/burning mouthfeel parameter, the proportion of cyclodextrin had a greater effect. At the higher level of cyclodextrin, the tongue sting/burning mouthfeels were at the level of patient perception for the 1 mg/mL dasotraline strength and eliminated at the 0.5 mg/mL strength, as can be seen in FIG. 4 (formulations 12 and 13) and FIG. 5 (formulations 10 and 11). The effects of cyclodextrin on cooling were similar to those seen in the tongue sting/burning parameter: At the higher cyclodextrin level, cooling was at the level of patient perception for the 1 mg/mL dasotraline strength and effectively eliminated at the 0.5 mg/mL strength [FIG. 6 (formulations 12 and 13) and FIG. 7 (formulations 10 and 11)]. Cyclodextrin was effective in reducing glycyrrhiza mouthfeel but the effects at the higher usage level were small. No aromatic off-notes were observed in any of the dasotraline solutions. This represents an improvement over the formulation of Example 1, which was characterized by patient-perceptible metallic and dairy sour aromatic offnotes. Numbing and tannin mouthfeels, not observed in Example 1, were at the threshold of patient perception in formulations 10-13. Astringency was also perceived in formulations 10-13, but more pronounced at the lower cyclodextrin strengths. It is possible that these mouthfeels were present in Example 1 but were overpowered by the strong intensity of the other aversive mouthfeels (e.g. tongue sting/mouth burn). By a unique combination of excipients, formulations 10-13 reduced the perception of the negative attributes of dasotraline to near or below the threshold of patient perception.

While not explicitly needed to mask the negative attributes of dasotraline—which are addressed by the formulations described above—it may be desirable in some instances to include an identifying flavor (e.g. orange, cherry, grape) to contribute to patient adherence.

### Example 9: Photostability of Pharmaceutical Compositions Comprising Dasotraline

Photostability of pharmaceutical compositions comprising dasotraline was evaluated. Samples were prepared by mixing components shown in Table 5. After filtration, each sample was filled into glass vials. Samples were stored at 60° C. up to 4 weeks, and 40° C. and 25° C. up to 6 months. The samples were evaluated after 0.6 million lux·hr and 1.2 million lux·hr. Samples that were protected from light showed good stability when stored at 40° C. and 25° C. for up to 6 months. However, an increase in the amount of unspecified impurities was observed in samples after exposure to light (1.2 million lux·hr).

TABLE 5
Pharmaceutical Compositions Comprising Dasotraline

| | Sweetening | Buffering | | |
|---|---|---|---|---|
| Cyclodextrin | Agent | agent | Preservative | Dasotraline |
| 1% HPBC | 0.01% | 25 mM | 0.2% | 1.0 mg/mL |
| 2.5% HPBC | Sucralose | Malic | Benzoic acid | |
| 0.5% HPBC | | acid | | 0.5 mg/mL |
| 1.25% HPBC | | | | |

1. A pharmaceutical composition for oral administration comprising dasotraline, or a pharmaceutically acceptable salt thereof, and one or more pharmaceutically acceptable excipients, wherein the pharmaceutical composition provides a decreased bitterness or paresthesic sensation.

2. The pharmaceutical composition of claim 1, wherein the one or more pharmaceutically acceptable excipients are each independently buffering agents, preservatives, or taste-masking agents.

3. The pharmaceutical composition of claim 1, wherein the pharmaceutical composition is chemically stable.

4. The pharmaceutical composition of claim 1, wherein the pharmaceutical composition is resistant to microbial growth.

5. The pharmaceutical composition of claim 2 wherein the buffering agent is citric acid, tartaric acid, malic acid, acetic acid, lactic acid, phosphoric acid, maleic acid, sodium citrate, sodium tartrate, sodium malate, sodium acetate, sodium lactate or sodium dihydrogen phosphate dehydrate.

6. The pharmaceutical composition of claim 2 wherein the buffering agent is malic acid.

7. The pharmaceutical composition of claim 2 wherein the preservative is benzoic acid, sodium benzoate, methyl paraben, propyl paraben, propylene glycol, sorbic acid, potassium sorbate or sodium dehydroacetate.

8. The pharmaceutical composition of claim 2 wherein the preservative is benzoic acid.

9. The pharmaceutical composition of claim 2 wherein the taste-masking agent is cyclodextrin, or a derivative thereof.

10. The pharmaceutical composition of claim 2 wherein the taste-masking agent is cyclodextrin is α-cyclodextrin, β-cyclodextrin, γ-cyclodextrin, methyl-β-cyclodextrin, hydroxypropyl-β-cyclodextrin, sulfobutylether-β-cyclodextrin sodium salt, randomly methylated β-cyclodextrin, branched β-cyclodextrins or hydroxypropyl-γ-cyclodextrins.

11. The pharmaceutical composition of claim 2 wherein the taste-masking agent is β-cyclodextrin.

12. The pharmaceutical composition of claim 2 wherein the taste-masking agent is hydroxypropyl-β-cyclodextrin.

13. The pharmaceutical composition of claim 2 wherein the taste-masking agent is cyclodextrin, or a derivative thereof and optionally a sweetening agent selected from the group consisting of aspartame, saccharin calcium, dextrose, fructose, maltitol, mannitol, saccharin, saccharin sodium, sorbitol, sucralose, sucrose, syrup, and acesulfame potassium.

14. The pharmaceutical composition of claim 13, wherein the optional sweetening agent is sucralose.

15. The pharmaceutical composition of claim 1 wherein the pharmaceutically acceptable salt is hydrochloride.

16. A method for decreasing bitterness or paresthesic sensation of a pharmaceutical composition comprising dasotraline, or a pharmaceutically acceptable salt thereof, by preparing a pharmaceutical composition according to claim 12.

17. A method of masking an undesirable taste or paresthesic sensation of a pharmaceutical composition comprising dasotraline, or a pharmaceutically acceptable salt thereof, by preparing a pharmaceutical composition according to claim 12.

18. A method of treating a disorder while decreasing bitterness or paresthesic sensation comprising administering a liquid pharmaceutical composition comprising dasotraline, or a pharmaceutically acceptable salt thereof, and one or more pharmaceutically acceptable excipients, wherein the administration comprises:
(a) measuring an amount of the pharmaceutical composition with a cup, a dosing spoon, or a dropper, or an oral syringe; and
(b) delivering the measured amount of the pharmaceutical composition to a patient in need thereof.

19. A flavoring system of a pharmaceutical composition comprising dasotraline, or a pharmaceutically acceptable salt thereof, wherein the pharmaceutical composition comprises one or more buffering agents, preservatives, or taste-masking agents.
